(19) United States
(12) Patent Application Publication
(10) Pub. No.: US 2009/0035328 A1
(43) Pub. Date: Feb. 5, 2009

(54) fHbp- AND LPXL1-BASED VESICLE VACCINES FOR BROAD SPECTRUM PROTECTION AGAINST DISEASES CAUSED BY NEISSERIA MENINGITIDIS

(76) Inventor: Dan Granoff, Berkeley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/185,021

(22) Filed: Aug. 1, 2008

Related U.S. Application Data
(60) Provisional application No. 60/953,674, filed on Aug. 2, 2007.

(51) Int. Cl. A61K 39/095; C12P 21/00; A61P 31/04

(52) U.S. Cl. 424/200.1; 435/71.2; 435/69.3

(57) ABSTRACT

The present invention generally provides methods and compositions for eliciting an immune response against Neisseria spp. bacteria in a subject, using vesicle vaccines made from Neisseria strains have decreased or no detectable expression of a product of LpxL1 gene, and which optionally overexpress fHbp.

### CROSS-REFERENCE TO RELATED APPLICATION

This application claims priority benefit of U.S. provisional application Ser. No. 60/953,674, filed Aug. 2, 2007, which application is incorporated herein by reference in its entirety.

### STATEMENT REGARDING FEDERALLY SPONSORED RESEARCH

This invention was made with government support under Public Health Service grant nos. RO1 AI46464, R21AI061533, from the National Institute of Allergy and Infectious Diseases of the National Institutes of Health, and T32-HL007951, from the National Heart, Lung and Blood Institute of the National Institutes of Health. The government has certain rights in this invention.

### TECHNICAL FIELD

This invention relates to broad-spectrum vaccines for diseases caused by Neisseria meningitidis.

### BACKGROUND

Neisseria meningitidis is a Gram-negative bacterium which colonizes the human upper respiratory tract and is responsible for worldwide sporadic and cyclical epidemic outbreaks of, most notably, meningitis and sepsis. The attack and morbidity rates are highest in children under 2 years of age. Like other Gram negative bacteria, Neisseria meningitidis typically possess a cytoplasmic membrane, a peptidoglycan layer, an outer membrane which together with the capsular polysaccharide constitute the bacterial wall, and pili which project into the outside environment. Encapsulated strains of Neisseria meningitidis are a major cause of bacterial meningitis and septicemia in children and young adults (Rosenstein et al. J Infect Dis 1999; 180:1894-901).

Humans are the only known reservoir for Neisseria meningitidis spp. Accordingly, Neisseria have evolved a wide variety of highly effective strategies to evade the human immune system. These include expression of a polysaccharide capsule that is structurally identical with host polysialic acid (i.e. serogroup B) and high antigenic mutability for the immunodominant noncapsular epitopes, i.e. epitopes of antigens that are present at the surface in relatively large quantities, are accessible to antibodies, and elicit a strong antibody response.

Outer membrane vesicle (OMV) vaccines have been proven to elicit protective immunity against group B meningococcal disease in humans (reviewed in Jodar et al. Lancet 2002; 359:1499-1508). Recently an OMV vaccine was licensed and introduced in New Zealand in response to a public health intervention to halt a group B epidemic that has been ongoing for more than a decade (Thomas et al. N Z Med J 2004; 117:U1016; Desmond et al. Nurs N Z 2004; 10:2; Baker et al. J Paediatr Child Health 2001; 37:S13-9). Other vesicle-based approaches to immunization have been described (see, e.g., Cartwright K et al, 1999, Vaccine; 17:2612-2619; de Kleinjn et al, 2000, Vaccine, 18:1456-1466; Rouupe van der Voort ER, 2000, Vaccine, 18:1334-1343; Tappero et al., 1999, JAMA 281:1520; Rouupe van der Voort ER, 2000, Vaccine, 18:1334-1343;US 2002/0110569; WO 02/09643).

N. meningitidis strains can be subdivided into three fHbp variant groups (v.1, v.2, and v.3) based on amino acid sequence variability and immunologic cross-reactivity (Masignani et al. J Exp Med 2003; 197:789-99). Variant 1 strains account for about 60% of disease-producing group B isolates (Masignani et al. 2003, supra). Within each variant group, there is on the order of about 92% or greater conservation of amino acid sequence.

Mice immunized with recombinant fHbp developed high serum bactericidal antibody responses against strains expressing fHbp proteins of the homologous variant group (Masignani et al. 2003, supra; Welsch et al. 2004, supra). However, a number of strains that expressed sub-variants of the respective fHbp protein were resistant to anti-fHbp complement-mediated bacteriolysis. Although the cause of this phenomenon is not known, conceivably this may be due to minor fHbp polymorphisms, or due to strain differences in the accessibility of critical fHbp epitopes on the surface of the bacteria that result in decreased binding and/or complement activation by the anti-fHbp antibodies. The recombinant fHbp protein vaccine used in the above immunogenicity studies was expressed in E. coli as a His-Tag protein devoid of the leader peptide. The recombinant protein also lacked the motif necessary for post-translational lipidation, which may decrease immunogenicity (Fletcher et al. Infect Immun 2004; 72: 2088-100).

Because they are made directly from N. menintigitidis strains, vesicle-based vaccines such as OMV vaccines contain endotoxin (LPS). However, removal of endotoxin can result in a reduction in immunogenicity. Thus another challenge is to develop a vesicle-based vaccine that is reduced in toxicity relative to wildtype N. menintigitidis strains (e.g., clinical strains) while retaining immunogeniticity and the ability to elicit bactericidal antibodies following administration to a host in need of vaccination.

### SUMMARY

The present disclosure generally provides methods and compositions for eliciting an immune response against Neisseria bacteria in a subject, using vesicle vaccines made from Neisseria strains with decreased or no detectable expression of a product of LpxL1 gene, and which optionally overexpress fHbp.

In one aspect, a composition comprising isolated antigenic vesicles prepared from a first Neisseria bacterium, wherein the Neisseria bacterium is genetically modified to provide for decreased or no activity of a gene product of the lpxL1 gene and express a heterologous fHbp polypeptide, an isolated Neisserial antigen and a pharmaceutically acceptable carrier is provided. In some aspects, the isolated Neisserial antigen comprises fHbp, GNA2132 and Nad A polypeptides. The isolated Neisserial antigen may further comprise GNA2091 and GNA1030 polypeptides. The heterologous fHbp can be fHbp v.2. The first Neisseria bacterium can be NZ98/254.

In some embodiments, the composition further comprises isolated antigenic vesicles prepared from a second Neisseria bacterium genetically diverse to the first Neisseria bacterium, wherein the second Neisseria bacterium is genetically modified to provide for decreased or no activity of a polypeptide product of the lpxL1 gene and provide for decreased or no production of an endogenous fHbp polypeptide and express a recombinant fHbp polypeptide. In some embodiments, the recombinant fHbp polypeptide is expressed from a construct comprising a nucleic acid encoding a fHbp polypeptide operably linked to a heterologous promoter. In other aspects, the recombinant fHbp polypeptide is of the same variant group as fHbp polypeptide endogenous to the second Neisseria bacterium. In some embodiments, the recombinant fHbp polypeptide is of the same variant group as fHbp polypeptide endogenous to the second Neisseria bacterium and expressed from a construct comprising a nucleic acid encoding the fHbp polypeptide operably linked to a heterologous promoter. In some aspects, the second bacterium is H44/76. In some embodiments, the first Neisseria bacterium is NZ98/254 and the second Neisseria bacterium is H44/76.

In some embodiments, the disclosure provides compositions comprising isolated antigenic vesicles prepared from a first Neisseria bacterium that is genetically modified to provide for decreased or no activity of a polypeptide product of the lpxL1 gene, isolated antigenic vesicles prepared from a second Neisseria bacterium that is genetically modified to provide for decreased or no activity of a polypeptide product of the lpxL1 gene and is genetically diverse to the first Neisseria bacterium, and a pharmaceutically acceptable carrier. In a related embodiment, the composition further comprises Nesisserial antigens comprising fHbp, GNA2132 and Nad A polypeptides. The isolated Neisserial antigen may further comprise GNA2091 and GNA1030 polypeptides. The first Neisseria bacterium can be NZ98/254 and the second Neisseria bacterium can be H44/76.

In certain embodiments, the composition comprises of a first Neisseria bacterium that is genetically modified to provide for decreased or no activity of a polypeptide product of the lpxL1 gene and to express a heterologous fHbp polypeptide and a second Neisseria bacterium that is genetically modified to provide for decreased or no activity of a polypeptide product of the lpxL1 gene, decreased production of endogenous fHbp polypeptide and for expression of a recombinant fHbp polypeptide. The recombinant fHbp polypeptide is expressed from a construct comprising a nucleic acid encoding a fHbp polypeptide operably linked to a heterologous promoter. The recombinant fHbp polypeptide is of the same variant group as fHbp polypeptide endogenous to the second Neisseria bacterium. In certain aspects, the first Neisseria bacterium can be NZ98/254 and the second Neisseria bacterium can be H44/76.

In another aspect, the present disclosure features a method of eliciting an immune response against Neisseria, the method comprising the steps of administering to a mammal an immunologically effective amount of any of the above compositions, wherein said administering is sufficient to elicit an immune response to a fHbp polypeptide present in the composition. Accordingly, a method of eliciting an immune response against Neisseria, comprises administering to a mammal an immunologically effective amount of the composition comprising antigenic vesicles prepared from a first Neisseria bacterium, wherein the Neisseria bacterium is genetically modified to provide for decreased or no activity of a gene product of the lpxL1 gene and express a heterologous fHbp polypeptide, an isolated Neisserial antigen, wherein said administering is sufficient to elicit an immune response to a fHbp polypeptide present in the composition.

In another aspect, the present disclosure features methods of producing antigenic compositions from any of the compositions disclosed herein. As such a method of producing an antigenic composition comprising culturing a Neisseria bacterium, wherein the Neisseria bacterium is genetically modified to provide for decreased or no activity of a gene product of the lpxL1 gene, and express a heterologous fHbp polypeptide, preparing isolated vesicles from the cultured bacterium, combining the isolated vesicles with an isolated Neisserial antigen and a pharmaceutically acceptable carrier, wherein an antigenic composition is produced. The isolated Nesisserial antigen comprises fHbp, GNA2132 and Nad A. In some embodiments, the isolated Nesisserial antigen further comprises GNA2091 and GNA1030 polypeptides. In another embodiment, the instant disclosure provides a method of producing the antigenic composition, the method comprising culturing a first Neisseria bacterium that is genetically modified to provide for decreased or no activity of a polypeptide product of the lpxL1 gene, culturing a second Neisseria bacterium that is genetically modified to provide for decreased or no activity of a polypeptide product of the lpxL1 gene and is genetically diverse to the first Neisseria bacterium, and preparing vesicles from the cultured first Neisseria bacterium and second Neisseria bacterium, combining the vesicles with a pharmaceutically acceptable carrier, wherein an antigenic composition is produced. In some embodiments, the first Neisseria bacterium is NZ98/254 and the second Neisseria bacterium is H44/76. In another aspect, the method of producing antigenic composition further comprises culturing a first Neisseria bacterium that is genetically modified to provide for decreased or no activity of a gene product of the lpxL1 gene and to express a heterologous fHbp polypeptide, and a second Neisseria bacterium that is genetically modified to provide for decreased or no activity of a gene product of the lpxL1 gene, decreased production of endogenous fHbp polypeptide and expression of a recombinant fHbp polypeptide, preparing vesicles from the cultures, combining vesicles with Nesisserial antigen comprising fHbp, GNA2132 and Nad A. In certain embodiments, this composition is combined with Nesisserial antigen comprising GNA2091 and GNA1030 polypeptides

### BRIEF DESCRIPTION OF THE DRAWINGS

FIG. 1 is a summary of strains tested for serum bactericidal activity (SBA) in Examples 1 and 2.

FIG. 2 shows the results of SDS-PAGE silver stained analysis of LPS produced from a wildtype H44/76 and from a genetically modified H44/76 having knockouts in LpxL1 and fHbp, and over-expressing fHbp (LpxL1KO, OE fHbp).

FIG. 3 is a graph showing dose-response release of proinflammatory cytokines IL-1β, -α after incubation of PBMCs with different concentrations of MV for 4 hours. Left, peripheral blood mononuclear cells (PBMCs) from human Donor 1, experiment 1. Right, PBMCs from human Donor 2, experiment 2. MV vaccines tested were native OMV prepared from the H44/76 wildtype (WT native, open squares with solid line) or a H44/76 mutant with inactivated LpxL1 and fHbp and over-expressed fHbp (mutant, native, closed squares with solid line), or a detergent-extracted MV from H44/76 wildtype strain (WT extracted, open circles with dashed lines).

FIG. 4 shows Western blot analysis of fHbp v.1 in MV vaccines produced from a wildtype H44/76, a mutant of H44/76 in which the gene encoding fHbp had been inactivated (fHbp KO), and from a genetically modified H44/76 having knockouts in LpxL1 and fHbp, and over-expressing fHbp (LpxL1KO, OE fHbp). The detecting antibody is anti-fHbp mAb, JAR 3, specific fHbp v.1. Amounts of MV loaded in each lane were standardized based on total protein content.

FIG. 5 shows that robust serum antibody response to factor H-binding protein variant 1 (fHbp v.1) was observed, as measured by ELISA. Left to right, mice immunized with aluminium hydroxide (Al(OH)₃); the Norwegian OMV vaccine (Norw. OMV); a recombinant protein vaccine (r5CV); or native OMV prepared from H44/76 with inactivated LpxL1 and fHbp and increased expression of fHbp (LpXL1 OE fHbp MV). The error bars show the 95% confidence intervals of the geometric mean titers.

FIG. 6 summarizes the geometric means of the serum bactericidal titers as measured against seven test strains. The titers of mice immunized with the aluminium hydroxide-adsorbed MV vaccine prepared from the double LpxL1/fHbp KO mutant with overexpressed fHbp are compared to the respective titers of mice immunized with the 5C recombinant protein or the Norway detergent-extracted OMV vaccines, each administered with aluminium hydroxide. Strain H44/76 (ET 5/ST 32) (B:15:P1.7, 16) was used to prepare the microvesicle (MV) vaccines and expresses a homologous v.1 fHbp to that of the recombinant protein. The remaining six strains have heterologous PorA molecules to that of the strain used to prepare the vesicle vaccine and also express subvariants of variant 1 fHbp as compared to fHbp expressed by H44/76 or the mutant strain with overexpressed fHBP.

FIG. 7 shows that the engineered LpxL1KO mutant of NZ98/254 expresses the endogenous fHbp v.1 (detected with anti-fHbp mAb JAR 5) and the heterologous fHbp v.2 encoded by the gene from strain 8047 (the protein was detected with anti-fHbp mAb JAR 11). For description of the anti-fHbp mAbs, see Beernink P. T. and Granoff D. M. Infect Immun. 2008, 76(6):2568-75. 8047 MV was prepared from wildtype (WT) N. meningitidis strain 8047 expressing fHbp in the v.2 group. KO, v.2 MV is from the strain in which the fHbp gene was inactivated. NZ98/254 LpxL1KO, MV from mutant of strain NZ98/254 in which the LpxL1 gene involved in lipopolysaccharide biosynthesis has been inactivated. Mutant v.1+v.2, MV is from the LpxL1 KO mutant of NZ98/254 that expresses endogenous fHbp (subvariant of v.1) and heterologous fHbp in the v.2 group (gene from 8047). rfHbp, purified His-tag recombinant protein controls expressed from E. coli (v.1, encoded by gene from N. meningitidis MC58; v.2, encoded by gene from strain 8047).

FIG. 8A shows IgG antibody responses of mice immunized with monovalent native MV vaccines from LpxL1 KO mutants. Antigen on the plate was recombinant fHbp variant 1 (v.1) or fHbp variant 2 (v.2). The bars represent the geometric mean titers of 2 or 3 serum pools (each pool contained serum samples from 4-5 mice). Native MV vaccines were prepared from LpxL1 knockout mutants of NZ98/254 that expressed only endogenous fHbp (subvariant of v.1; designated “NZ mutant*”); or that expressed both endogenous fHbp v.1, and a heterologous v.2 encoded by the gene from strain 8047 (designated “NZ fHbp mutant**”); or a LpxL1 KO mutant of strain H44/76 in which the gene encoding endogenous fHbp also is inactivated and with over-expressed fHbp v.1, encoded by the gene from H44/76 (designated “H44/76 fHbp mutant***”). The “r3C” contained recombinant fHbp v.1 (encoded by gene from strain MC58), GNA2132 (encoded by gene from strain NZ98/254), and NadA (encoded by the gene from strain 2996). A group of mice received A1 (OH)₃ only as a negative control (all of the MV and recombinant proteins in the vaccine groups were adsorbed with A1(OH) 3).

FIG. 8B shows IgG antibody responses of mice immunized with bivalent native MV vaccines prepared from LpxL1 KO mutants. See legend to FIG. 7A. Bivalent NZ and H44/76 MV vaccines: Detergent WT, detergent extracted OMVs from wildtype strains of NZ98/254 and H44/76; Native mutants*, MV prepared from LpxL1 KO mutants of NZ98/254 and H44/76 that that expressed only the endogenous fHbps in the v.1 group; Native fHbp mutants**, MV from LpxL1 KO mutants of strain NZ98/254 that expressed both endogenous fHbp (subvariant of v.1 group), and heterologous fHbp v.2 (encoded by gene from 8047) and MV from a LpxL1 mutant of H44/76 that over-expressed fHbp v.1 (gene from H44/76); r3C, see legend to FIG. 7A; rfHBp v.2, recombinant fHbp v.2. The geometric mean of the anti-fHbp v.1 antibody responses of mice that received the bivalent MV vaccine from the native fHbp mutants** was significantly higher than that of group immunized with the vaccine from the native mutants* that expressed only endogenous fHbp (P<0.05).

FIG. 9 shows serum bactericidal antibody responses of mice measured against the homologous wildtype strains used to prepare the mutants for the MV vaccines. The bars represent the geometric mean titers measured in 2 or 3 serum pools from each group (each pool contained serum samples from 4-5 mice). Titers were measured with human complement. For vaccine groups see legends to FIGS. 8A and 8B.

FIG. 10 shows reverse cumulative distribution of serum bactericidal antibody responses of mice immunized with monovalent MV vaccines (Panel A) or control recombinant protein vaccines (Panel B) as measured against group B N. meningitidis strains with heterologous PorA to those of the vaccine strains, and fHbp in the v.2 or v.3 groups (N=6, see Table 3). Titers beginning at a serum dilution of 1:10 were measured with human complement. Susceptibility of a strain and endpoint titer was defined by the highest dilution of serum that elicited a 50 percent decrease in CFU/ml after incubation of bacteria with the test serum and 20% complement for 60 minutes, as compared with CFU in control sera at time 0. For vaccine groups see FIGS. 8A and 8B. Detergent WT+3C denotes detergent-treated MV from WT strain+three recombinant protein (3C).

FIG. 11 shows reverse cumulative distribution of serum bactericidal antibody responses of mice immunized with bivalent MV vaccines (Panel A) or control recombinant protein vaccines (Panel B) as measured against eleven N. meningitidis strains from the Unites States or Europe that had heterologous PorA to those of the vaccine strains, and fHbp in the v.1 (N=5 strains), v.2 (N=4) or v.3 group (N=2) (See list of test strains in Table 3). Titers were measured with human complement. For vaccine groups see FIGS. 8A and 8B. Susceptibility of a strain was defined as a decrease of 50 percent in the CFU/ml when incubated for 1 hr at 37 degrees with 20 percent human complement and different dilutions of test sera, as compared with the respective CFU/ml at time 0 in negative control sera and complement.

FIG. 12 shows the geometric mean bactericidal titers of sera from mice immunized with bivalent MV vaccines as measured against epidemic group A, W-135 and X strains from Africa. All of the strains had heterologous PorA to those of the strains used to prepare the MV vaccines. All of the strains except the ST-11 W-135 strain from Burkina Faso expressed subvariants of fHbp v.1 group (the strain from Mali expressed a subvariant of fHbp v.2).

Before the present invention and specific exemplary embodiments of the invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an antigen” includes a plurality of such antigens and reference to “the vesicle” includes reference to one or more vesicles and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF EXEMPLARY EMBODIMENTS

The present disclosure generally provides methods and compositions for eliciting an immune response against Neisseria bacteria in a subject, using vesicle vaccines made from Neisseria strains having decreased or no detectable expression of a product of LpxL1 gene, and which optionally overexpress fHbp.

The examples provided herein illustrate the breadth of protection elicited by immunization with a vesicle vaccine (exemplified by an MV vaccine) prepared from a N. meningitidis strain genetically modified to provide for decreased or no activity of the product of the LpxL1 gene and optionally over-expressing fHbp. Functional activities of the anti-fHbp antibodies elicited by the vesicle vaccine from strains genetically modified to provide for decreased or no activity of the product of the lpxL1 gene and expressing fHbp were greater than that of the antibodies elicited by the recombinant fHbp vaccine, or a combination of recombinant fHbp and vesicles prepared from the wildtype strain.

### DEFINITIONS

The term “protective immunity” means that a vaccine or immunization schedule that is administered to a mammal induces an immune response that prevents, retards the development of, or reduces the severity of a disease that is caused by Neisseria meningitidis, or diminishes or altogether eliminates the symptoms of the disease.

The phrase “a disease caused by a strain of serogroup B of Neisseria meningitidis” encompasses any clinical symptom or combination of clinical symptoms that are present in an infection with a member of serogroup B of Neisseria meningitidis. These symptoms include but are not limited to: colonization of the upper respiratory tract (e.g. mucosa of the nasopharynx and tonsils) by a pathogenic strain of serogroup B of Neisseria meningitidis, penetration of the bacteria into the mucosa and the submucosal vascular bed, septicemia, septic shock, inflammation, haemorrhagic skin lesions, activation of fibrinolysis and of blood coagulation, organ dysfunction such as kidney, lung, and cardiac failure, adrenal hemorrhaging and muscular infarction, capillary leakage, edema, peripheral limb ischaemia, respiratory distress syndrome, pericarditis and meningitis.

The phrase “broad spectrum protective immunity” means that a vaccine or immunization schedule elicits “protective immunity” against at least one or more (or against at least two, at least three, at least four, at least five, against at least eight, or at least against more than eight) strains of Neisseria meningitidis, wherein each of the strains belongs to a different serosubtype as the strains used to prepare the vaccine. The present disclosure specifically contemplates and encompasses a vaccine or vaccination regimen that confers protection against a disease caused by a member of serogroup B of Neisseria meningitidis and also against other serogroups, particularly serogroups A, C, Y and W-135.

The phrase “specifically binds to an antibody” or “specifically immunoreactive with”, when referring to an antigen such as a polysaccharide, phospholipid, protein or peptide, refers to a binding reaction which is based on and/or is probative of the presence of the antigen in a sample which may also include a heterogeneous population of other molecules. Thus, under designated immunoassay conditions, the specified antibody or antibodies bind(s) to a particular antigen or antigens in a sample and do not bind in a significant amount to other molecules present in the sample. Specific binding to an antibody under such conditions may require an antibody or antiserum that is selected for its specificity for a particular antigen or antigens.

The phrase “in a sufficient amount to elicit an immune response to epitopes present in said preparation” means that there is a detectable difference between an immune response indicator measured before and after administration of a particular antigen preparation. Immune response indicators include but are not limited to: antibody titer or specificity, as detected by an assay such as enzyme-linked immunoassay (ELISA), bactericidal assay, flow cytometry, immunoprecipitation, Ouchter-Lowny immunodiffusion; binding detection assays of, for example, spot, Western blot or antigen arrays; cytotoxicity assays, etc.

A “surface antigen” is an antigen that is present in a surface structure of Neisseria meningitidis (e.g. the outer membrane, inner membrane, periplasmic space, capsule, pili, etc.).

The phrase “genetically diverse” as used in the context of genetically diverse strains of Neisseria meningitidis, refers to strains that differ from one another in the amino acid sequence of at least one, and usually at least two, more usually at least three polypeptides, particularly antigenic polypeptides. Genetic diversity of strains can be accomplished by selecting strains that differ in at least one or more, preferably at least two or more, of serogroup, serotype, or serosubtype (e.g., two strains that differ in at least one of the proteins selected from outer membrane, PorA and PorB proteins, are said to genetically diverse with respect to one another). Genetic diversity can also be defined by, for example, multi-locus sequence typing and/or multi-locus enzyme typing (see, e.g., Maiden et al., 1998, Proc. Natl. Acad. Sci. USA 95:3140; Pizza et al. 2000 Science 287:1816), multi-locus enzyme electrophoresis, and other methods known in the art.

“Serogroup” or “capsular group” as used herein refers to classification of Neisseria meningitides by virtue of immunologically detectable variations in the capsular polysaccharide. About 12 serogroups are known: A, B, C, X, Y, Z, 29-E, W-135, H, I, K and L. Any one serogroup can encompass multiple serotypes and multiple serosubtypes.

“Serotype” as used herein refers to classification of Neisseria meningitides strains based on monoclonal antibody defined antigenic differences in the outer membrane protein Porin B. A single serotype can be found in multiple serogroups and multiple serosubtypes.

“Serosubtype” as used herein refers classification of Neisseria meningitides strains based on antibody defined antigenic variations on an outer membrane protein called Porin A, or upon VR typing of amino acid sequences deduced from DNA sequencing (Sacchi et al., 2000, J. Infect. Dis. 182:1169; see also the Multi Locus Sequence Typing web site). Most variability between PorA proteins occurs in two (loops I and IV) of eight putative, surface exposed loops. The variable loops I and IV have been designated VR1 and VR2, respectively. A single serosubtype can be found in multiple serogroups and multiple serotypes.

“Enriched” means that an antigen in an antigen composition is manipulated by an experimentalist or a clinician so that it is present in at least a three-fold greater concentration by total weight, usually at least 5-fold greater concentration, more preferably at least 10-fold greater concentration, more usually at least 100-fold greater concentration than the concentration of that antigen in the strain from which the antigen composition was obtained. Thus, if the concentration of a particular antigen is 1 microgram per gram of total bacterial preparation (or of total bacterial protein), an enriched preparation would contain at least 3 micrograms per gram of total bacterial preparation (or of total bacterial protein).

The term “endogenous” refers to a naturally-occurring biological component of a cell, i.e., as found in nature.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to a coding sequence. “Heterologous” as used herein in the context of genes or proteins denotes genes or proteins that are naturally expressed in two different bacterial strains. For example, a first Neisserial strain expressing PorA 1.5-2, 10 and a second Neisserial strain expressing PorA 7-2, 4 are said to have “heterologous PorA proteins” or are “heterologous with respect to PorA”. Genes and proteins are also said to be “heterologous” where they expressed in the same strain, but are of different origin. For example, a strain that expresses an endogenous fHbp polypeptide and also expresses a recombinant fHbp that differs in amino acid sequence from the endogenous fHbp polypeptide (e.g., is of a different variant group or variant subtype) is said to contain “heterologous fHbp polypeptides”.

“Recombinant” as used herein refers to nucleic acid encoding a gene product, or a gene product (e.g., polypeptide) encoded by such a nucleic acid, that has been manipulated by the hand of man, and thus is provided in a context or form in which it is not found in nature. “Recombinant” thus encompasses, for example, a nucleic acid encoding a gene product operably linked to a heterologous promoter (such that the construct that provides for expression of the gene product from an operably linked promoter with which the nucleic acid is not found in nature). For example, a “recombinant fHbp” encompasses a fHbp encoded by a construct that provides for expression from a promoter heterologous to the fHbp coding sequence, fHbp polypeptides that are modified relative to a naturally-occurring fHbp (e.g., as in a fusion protein), and the like. It should be noted that a recombinant fHbp polypeptide can be endogenous to or heterologous to a N. meningitidis strain in which such a recombinant fHbp-encoding construct is present.

The term “immunologically naïve with respect to Neisseria meningitidis” denotes an individual (e.g., a mammal such as a human patient) that has never been exposed (through infection or administration) to Neisseria meningitidis or to an antigen composition derived from Neisseria meningitidis in sufficient amounts to elicit protective immunity, or if exposed, failed to mount a protective immune response. (An example of the latter would be an individual exposed at a too young age when protective immune responses may not occur. Molages et al., 1994, Infect. Immun. 62: 4419-4424). It is further desirable (but not necessary) that the “immunologically naïve” individual has also not been exposed to a Neisserial species other than Neisseria meningitidis (or an antigen composition prepared from a Neisserial species), particularly not to a cross-reacting strain of Neisserial species (or antigen composition). Individuals that have been exposed (through infection or administration) to a Neisserial species or to an antigen composition derived from that Neisserial species in sufficient amounts to elicit an immune response to the epitopes exhibited by that species, are “primed” to immunologically respond to the epitopes exhibited by that species.

A “knock-out” or “knockout” of a target gene refers to an alteration in the sequence of the gene that results in a decrease of function of the target gene, e.g., such that target gene expression is undetectable or insignificant, and/or the gene product is not function or not significantly functional. For example, a “knockout” of a gene involved in LPS synthesis indicates means that function of the gene has been substantially decreased so that the expression of the gene is not detectable or only present at insignificant levels and/or a biological activity of the gene product (e.g., an enzymatic activity) is significantly reduced relative to prior to the modification or is not detectable. “Knock-outs” encompass conditional knock-outs, where alteration of the target gene can occur upon, for example, exposure to a predefined set of conditions (e.g., temperature, osmolarity, exposure to substance that promotes target gene alteration, and the like.

A “monovalent vaccine” refers to a vesicle vaccine prepared from a single strain. The strain may be a mutant strain (i.e., genetically modified) or a wildtype strain (naturally occurring). Such vaccines may be combined with other immunogenic or antigenic components to provide a vaccine composition (e.g., combined with one or more recombinant protein antigens).

A “bivalent vaccine” refers to a vesicle vaccine prepared from two different strains. The two strains may be mutant strains or a wildtype strains or a combination of a mutant and a wildtype strain. Such vaccines may be combined with other immunogenic or antigenic components to provide an vaccine composition (e.g., combined with one or more recombinant protein antigens).

The term “native” when used in context of vesicles or vesicle vaccines refers to vesicles that are not detergent treated. These native vesicles may be obtained from, for example, naturally occurring strains that produce vesicles with low endotoxicity or from strains genetically modified to produce vesicles with low endotoxicity.

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

As noted above, fHbp has been divided into three variant groups (referred to as variant 1 (v.1), variant 2 (v.2), and variant 3 (v.3)) based on amino acid sequence variability and immunologic cross-reactivity (Masignani et al. 2003 J Exp Med 197:789-99). “Variant” as used in the context of an “fHbp variant” refers to an fHbp that share at least 89% amino acid sequence identity with the prototype strain of that variant group (strain MC58 for v.1; strain 2996 for v.2; and strain M1239 for v.3). These were the original prototype sequences described by Masignani et al., J. Exp. Med., 2003. Strains within a variant group encode fHbps with greater than 88% amino acid identity, whereas strains of different fHbp variant groups range from approximately 60-88% identical. fHbp in the same “variant” group possess greater than 88% identity to the respective prototype sequence (v.1, strain MC58; v.2, strain 2996; v.3, strain M1239). A “subvariant” as used in the context of an “fHbp subvariant” refers to fHbp polypeptides that differ from the prototype sequence. For example, strain NZ98/254 is referred to as an fHbp v.1 subvariant, with 91% identity to the prototype sequence from strain MC58; strain RM1090 is referred to as an fHBP v.2 subvariant, with a sequence that is 94% identical to the v.2 prototype strain 2996. It should be noted that “fHbp” is also referred to as GNA1870.

Neisserial Strains Expressing fHbp for Use in Vesicle Production

In general, the present disclosure involves production of vesicles (microvesicles or outer membrane vesicles) from Neisserial strain genetically modified to provide for decreased or no activity of the product of the lpxL1 gene and that produces a level of fHbp protein sufficient to provide for vesicles that, when administered to a subject, evoke serum anti-fHbp antibodies. The anti-fHbp antibodies produced facilitate immunoprotection against 1, 2, 3, 4, 5, 6, 7, 8, 9, 10 or more Neisserial strains, which strains can be genetically diverse (or “heterologous”) with respect to, for example, serogroup, serotype, serosubtype (e.g., as determined by PorA protein), Sequence type, electrophoretic type, fHbp variant, and/or fHbp subvariant.

Any of a variety of Neisseria spp. strains that produce or can be modified to produce fHbp, and, optionally, which produce or can be modified to produce other antigens of interest, such as PorA, GNA2132 etc., can be used in the methods of the present disclosure. Characteristics of suitable strains with respect to fHbp production are discussed in more detail below.

Pathogenic Neisseria spp. or strains derived from pathogenic Neisseria spp., particularly strains pathogenic for humans or derived from strains pathogenic or commensal for humans, are of particular interest. Exemplary Nessserial spp. include N. meningitidis, N. flavescens N. gonorrhoeae, N. lactamica, N. polysaccharea, N. cinerea, N. mucosa, N. subflava, N. sicca, N. elongata, and the like. “Derived from” in the context of bacterial strains is meant to indicate that a strain was obtained through passage in vivo, or in in vitro culture, of a parental strain and/or is a recombinant cell obtained by modification of a parental strain.

N. meningitidis strains are of particular interest in the present disclosure. N. meningitidis strains can be divided into serologic groups, serotypes and subtypes on the basis of reactions with polyclonal (Frasch, C. E. and Chapman, 1973, J. Infect. Dis. 127: 149-154) or monoclonal antibodies that interact with different surface antigens. Serogrouping is based on immunologically detectable variations in the capsular polysaccharide. About 12 serogroups (A, B, C, X, Y, Z, 29-E, and W-135) are known. Strains of the serogroups A, B, C, Y and W-135 account for nearly all meningococcal disease.

Serotyping is based on monoclonal antibody defined antigenic differences in an outer membrane protein called Porin B (PorB). Antibodies defining about 21 serotypes are currently known (Sacchi et al., 1998, Clin. Diag. Lab. Immunol. 5:348). Serosubtyping is based on antibody defined antigenic variations on an outer membrane protein called Porin A (PorA). Antibodies defining about 18 serosubtypes are currently known. Serosubtyping is especially important in Neisseria meningitidis strains where immunity may be serosubtype specific. Most variability between PorA proteins occurs in two (loops I and IV) of eight putative, surface exposed loops. The variable loops I and IV have been designated VR1 and VR2, respectively. Since more PorA VR1 and VR2 sequence variants exist that have not been defined by specific antibodies, an alternative nomenclature based on VR typing of amino acid sequence deduced from DNA sequencing has been proposed (Sacchi et al., 2000, J. Infect. Dis. 182:1169; see also the Multi Locus Sequence Typing web site). Lipopolysaccharides can also be used as typing antigens, giving rise to so-called immunotypes: L1, L2, etc.

N. meningitidis also may be divided into clonal groups or subgroups, using various techniques that directly or indirectly characterize the bacterial genome. These techniques include multilocus enzyme electrophoresis (MLEE), based on electrophoretic mobility variation of an enzyme, which reflects the underlying polymorphisms at a particular genetic locus. By characterizing the variants of a number of such proteins, genetic “distance” between two strains can be inferred from the proportion of mismatches. Similarly, clonality between two isolates can be inferred if the two have identical patterns of electrophoretic variants at number of loci. More recently, multilocus sequence typing (MLST) has superseded MLEE as the method of choice for characterizing the microorganisms. Using MLST, the genetic distance between two isolates, or clonality is inferred from the proportion of mismatches in the DNA sequences of 11 housekeeping genes in Neisseria meningitidis strains (Maiden et al., 1998, Proc. Natl. Acad. Sci. USA 95:3140).

The strain used for vesicle production can be selected according to a number of different characteristics that may be desired. For example, in addition to selection according to a level of fHbp production, the strain may be selected according to: a desired PorA type (a “serosubtype”, as described above), serogroup, serotype, and the like; decreased capsular polysaccharide production; and the like.

For example, the production strain can produce any desired PorA polypeptide, and may express one or more PorA polypeptides (either naturally or due to genetic engineering). Exemplary strains includes those that produce a PorA polypeptide which confers a serosubtype of P1.7, 16; P1.19, 15; P1.7, 1; P1.5, 2; P1.22a, 14; P1.14; P1.5, 10; P1.7, 4; P1.12, 13; as well as variants of such PorA polypeptides which may or may not retain reactivity with conventional serologic reagents used in serosubtyping.

Also of interest are PorA polypeptides characterized according to PorA variable region (VR) typing (see, e.g., Russell et al. Emerging Infect Dis 2004 10:674-678; Sacchi C T, et al, Clin Diagn Lab Immunol 1998; 5:845-55; Sacchi et al, J. Infect Dis 2000; 182:1169-1176). A substantial number of distinct VR types have been identified, which can be classified into VR1 and VR2 family “prototypes”. A web-accessible database describing this nomenclature and its relationship to previous typing schemes is found at neisseria.org/nm/typing/pora. Alignments of exemplary PorA VR1 and VR2 types is provided in Russell et al. Emerging Infect Dis 2004 10:674-678.

Exemplary PorA polypeptides as characterized by PorA serosubtypes include P1.5, 2; P1.5a, 2a; P1.5a, 2c; P1.5a, 2c; P1.5a, 2c; P1.5b, 10; P1.5b, 10; P1.5b, C; P1.7, 16; P1.7d, 1; P1.7d, 1; P1.7d, 1; P1.7d, 1; P1.7b, 3; P1.7b, 4; P1.7b, 4; P1.12, 16; P1.12a, 13a; P1.22, 9; P1.23, 14; P1.23, 14; P1.19, 15; P1.B, 1; P1.C, 1; P1.E, A; P1.E, A; P1.E, A; P1.5, 2; P1.5, 2; P1.5a, 10a; P1.5b, 10; P1.5b, 10; P1.5b, 10b; P1.7, 16; P1.7, 16; P1.7b, 1; P1.7b, 13e; P1.7b, 4; P1.7b, 4; P1.7d, 1; P1.7d, 1; P1.7b, 13a; P1.23, 3; P1.23, 3; P1.23, 3; P1.19, 15; P1.19, 1; P1.19, 15; P1.19, 15; P1.19, 15; P1.19, 15; P1.19, 15; P1.19, 15; P1.19, 15; P1.E, A; P1.E, A; P1.E, 16a; P1.E, 4a; P1.E, 4a; P1.Ea, 3; P1.Eb, 9; P1.Eb, 9; P1.Eb, 9; P1.Eb, 9; P1.Eb, 9; P1.F, 16; P1.7a, 1; P1.7b, 3; P1.7d, 1; P1.Ea, 3; P1.5b, 10; P1.5b, 10; P1.5b, 10; P1.5b, 10; P1.5b, 10; P1.5b, 10; P1.5b, 10b; P1.5b, 10; P1.22, 14a; P1.F, 16; P1.D, 2d; P1.D, 2; P1.D, 2d; P1.19c, 2c; P1.D, 10f; P1.A, 10e; P1.A, 10g; P1.A, 10; P1.19, 15; P1.19, 15; P1.19, 15; P1.19, 15; P1.7b, 16; P1.7, 16b; P1.7, 16; P1.19, 15; P1.Eb, 9; P1.5, 2e; P1.E, A; P1.7b, 13d; P1.Ea, 3; P1.7, 16b; P1.Ec, 1; P1.7b, 4; P1.7b, 4; P1.7, 9; P1.19, 15; P1.19, 15; P1.19, 15; P1.19, 15a; P1.19a, 15b; P1.19, 15; P1.5b, 16; P1.19b, 13a; P1.5, 16; P1.5, 2; P1.5, 2b; P1.7b, 16; P1.7, 16b; P1.7b, 3; P1.Ea, 3; P1.5a, 2c; P1.F, 16; P1.5a, 9; P1.7c, 10c; P1.7b, 13a; P1.7, 13a; P1.7a, 10; P1.20, 9; P1.22,B; P1.5b, del; P1.5b, 10; P1.7, 13a; P1.12a, 13f; P1.12a, 13; P1.12a, 13a; P1.12a, 13a; P1.12a, 13; P1.12a, 13; P1.E, 13b; P1.7b, 13a; P1.7b, 13; P1.5, 2; P1.5, 2; P1.Ea, 3; P1.22, 9; P1.5, 2; P1.5, 2; P1.19, 15; P1.5, 2; P1.12b, 13a; P1.5c, 10a; P1.7e, 16e; P1.B, 16d; P1.F, 16e; P1.F, 16e; P1.7b, 13e; P1.B, 16d; P1.7e, 16e; P1.7b, 13g; P1.B, 16f; P1.7, 16c; P1.22, 14b; P1.22, 14c; P1.7, 14; P1.7, 14; and P1.23, 14.

Amino acid sequences of exemplary PorA polypeptides are found at GenBank accession nos. X57182, X57180, U92941, U92944, U92927, U92931, U92917, U92922, X52995, X57184, U92938, U92920, U92921, U92929, U92925, U92916, X57178, AF051542, X57181, U92919, U92926, X57177, X57179, U92947, U92928, U92915, X57183, U92943, U92942, U92939, U92918, U92946, U92496, U97260, U97259, AF042541, U92923, AF051539, AF051538, U92934, AF029088, U92933, U97263, U97261, U97262, U92945, AF042540, U92935, U92936, U92924, AF029086, AF020983, U94958, U97258, U92940, AF029084, U92930, U94959, U92948, AF016863, AF029089, U92937, AF029087, U92932, AF029090, AF029085, AF051540, AF051536, AF052743, AF054269, U92495, U92497, U92498, U92499, U92500, U92501, U92502, U92503, AF051541, X12899, Z48493, Z48489, Z48485, Z48494, Z48487, Z48488, Z48495, Z48490, Z48486, Z48491, Z48492, X66478, X66479, X66477, X66480, X81110, X79056, X78467, X81111, X78802, Z14281/82, Z14273/74, Z14275/76, Z14261/62, Z14265/66, Z14277/78, Z14283/84, Z14271/72, Z14269/70, Z14263/64, Z14259/60, Z14257/58, Z14293/94, Z14291/92, Z14279/80, Z14289/90, Z14287/88, Z14267/68, Z14285/86, L02929, X77423, X77424, X77433, X77426, X77428, X77430, X77427, X77429, X77425, X77432, X77431, X77422, Z48024/25, Z48032/33, Z48020/21, Z48022/23, Z48028/29, Z48016/17, Z48012/13, Z48014/15, Z48018/19, Z48026/27, U31060, U31061, U31062, U31063, U31064, U31065, U31066, U31067, U93898, U93899, U93900, U93901, U93902, U93903, U93904, U93905, U93906, U93907, and U93908.

Alternatively or in addition, the production strain can be a capsule deficient strain. Capsule deficient strains can provide vesicle-based vaccines that provide for a reduced risk of eliciting a significant autoantibody response in a subject to whom the vaccine is administered (e.g., due to production of antibodies that cross-react with sialic acid on host cell surfaces). “Capsule deficient” or “deficient in capsular polysaccharide” as used herein refers to a level of capsular polysaccharide on the bacterial surface that is lower than that of a naturally-occurring strain or, where the strain is genetically modified, is lower than that of a parental strain from which the capsule deficient strain is derived. A capsule deficient strain includes strains that are decreased in surface capsular polysaccharide production by at least 10%, 20%, 25%, 30%, 40%, 50%, 60%, 75%, 80%, 85%, 90% or more, and includes strains in which capsular polysaccharide is not detectable on the bacterial surface (e.g., by whole cell ELISA using an anti-capsular polysaccharide antibody).

Capsule deficient strains include those that are capsule deficient due to a naturally-occurring or recombinantly-generated genetic modification. Naturally-occurring capsule deficient strains (see, e.g., Dolan-Livengood et al. J. Infect. Dis. (2003) 187(10):1616-28), as well as methods of identifying and/or generating capsule-deficient strains (see, e.g., Fisseha et al. (2005) Infect. Immun. 73(7):4070-4080; Stephens et al. (1991) Infect Immun 59(11):4097-102; Frosch et al. (1990) Mol. Microbiol. 1990 4(7):1215-1218) are known in the art.

Modification of a Neisserial host cell to provide for decreased production of capsular polysaccharide may include modification of one or more genes involved in capsule synthesis, where the modification provides for, for example, decreased levels of capsular polysaccharide relative to a parent cell prior to modification. Such genetic modifications can include changes in nucleotide and/or amino acid sequences in one or more capsule biosynthesis genes rendering the strain capsule deficient (e.g., due to one or more insertions, deletions, substitutions, and the like in one or more capsule biosynthesis genes). Capsule deficient strains can lack or be non-functional for one or more capsule genes.

Of particular interest are strains that are deficient in sialic acid biosynthesis. Such strains can provide for production of vesicles that have reduced risk of eliciting anti-sialic acid antibodies that cross-react with human sialic acid antigens, and can further provide for improved manufacturing safety. Strains having a defect in sialic acid biosynthesis (due to either a naturally occurring modification or an engineered modification) can be defective in any of a number of different genes in the sialic acid biosynthetic pathway. Of particular interest are strains that are defective in a gene product encoded by the N-acetylglucosamine-6-phosphate 2-epimerase gene (known as synX AAF40537.1 or siaA AAA20475), with strains having this gene inactivated being of especial interest. For example, in one embodiment, a capsule deficient strain is generated by disrupting production of a functional synX gene product (see, e.g., Swartley et al. (1994) J. Bacteriol. 176(5):1530-4).

Capsular deficient strains can also be generated from naturally-occurring strains using non-recombinant techniques, e.g., by use of bactericidal anti-capsular antibodies to select for strains that reduced in capsular polysaccharide.

Where the use of two or more strains are is involved (e.g., to produce antigenic compositions of vesicles from different strains, as discussed below in more detail), the strains can be selected so as to differ in on or more strain characteristics, e.g., to provide for vesicles that differ in PorA type and/or fHbp variant group.

fHbp Production in Neisserial Host Cells

In general as noted above, vesicles can be produced using a Neisserial strain genetically modified to provide for decreased or no activity of the product of the lpxL1 gene and that produces vesicles with sufficient fHbp protein that, when administered to a subject, provide for production of anti-fHbp antibodies.

In one embodiment, the Neisserial strains genetically modified to provide for decreased or no activity of the product of the lpxL1 gene used to produce vesicles are not genetically modified with respect to fHbp, i.e., the the Neisserial strains produce the endogenous fHbp.

In one embodiment, the Neisserial strains genetically modified to provide for decreased or no activity of the product of the lpxL1 gene used to produce vesicles according to the present disclosure can be strains that express a higher level of fHbp relative to strains that express no detectable or a low level of fHbp. RM1090 is an example of a strain that produces a low level of fHbp. Naturally occurring strains that produce fHbp at a level that is 1.5, 2, 2, 53, 3.5, 4, 4.5, 5, 5.5, 6, 6, 5, 7, 7.5, 8, 8.5, 9, 9.5,or 10-fold or greater over fHbp production in a low fHbp-producing strain, such as RM1090, are of particular interest. Examples of naturally-occurring strains that express a high level of fHbp include ST-32/ET-5 strains such as H44/76, Cu385 and MC58. For a discussion of strains that express low or undetectable levels of fHbp, intermediate levels of fHbp, or high levels of fHbp see Masignani et al. 2003, J Exp Med 197:789-199 AND WELSCH ET AL. 2008, J Infect Dis 2008; 197:1053-61.

In some embodiments, the strain produces a level of fHbp that is greater than that produced in RM1090, and can be at least 1.5, 2, 2, 5 3, 3.5, 4, 4.5, 5, 5.5, 6, 6, 5, 7, 7.5, 8, 8.5, 9, 9.5, or 10-fold or greater than that in RM1090. In another embodiment, the Neisserial strain, genetically modified to provide for decreased or no activity of the product of the lpxL1 gene, is further modified by recombinant or non-recombinant techniques to provide for a sufficiently high level of fHbp production. Such modified strains generally are produced so as to provide for an increase in fHbp production that is 1.5, 2, 2, 5 3, 3.5, 4, 4.5, 5, 5.5, 6, 6, 5, 7, 7.5, 8, 8.5, 9, 9.5, or 10-fold or greater over fHbp production in the unmodified parental cell or over fHbp production of the strain RM1090. Any suitable strain can be used in this embodiment, including strains that produce low or undetectable levels of fHbp prior to modification and strains that naturally produce high levels of fHbp relative to strains that express no detectable or a low level of fHbp.

Modified strains can be generated by non-recombinant techniques such as, for example, exposure to chemicals, radiation, or other DNA modifying or damaging agent, and the like. Modified strains having a desired protein expression profile, particularly with respect to fHbp production, can be identified through screening for strains producing a desired level of fHbp (e.g., an increased level of fHbp as compared to the unmodified parental strain or a low fHbp producer (such as RM1090), or a level similar to that of a strain that produces fHbp at acceptably high levels).

Alternatively, and more usually, modified strains are produced using recombinant techniques, usually by introduction of nucleic acid encoding a fHbp polypeptide or manipulation of an endogenous fHbp gene to provide for increased expression of endogenous fHbp.

Methods for determining fHbp production levels are known in the art. Such methods include, for example, Western blot (optionally with analysis assisted by densitometry scan), flow cytometric (e.g., FACS) analysis using anti-fHbp antibody, detection of fHbp RNA levels, and the like. Strains that have higher levels of fHbp production, either naturally or due to genetic modification, are sometimes referred to herein as fHbp “over-expressers” or are said to “overexpress” fHbp. In general, where a strain is genetically modified to overexpress fHbp, the strain expresses a higher level of fHbp relative to the parent strain (prior to genetic modification).

### Production of Genetically Modified Neisserial Strains

As noted above, by introduction of nucleic acid encoding a fHbp polypeptide or manipulation of an endogenous fHbp gene to provide for increased expression of endogenous fHbp.

Neisserial Host Cells Genetically Modified to Provide for Increased Expression of an Endogenous fHbp

Endogenous fHbp expression can be increased by altering in situ the regulatory region controlling the expression of fHbp. Methods for providing for increased expression of an endogenous Neisserial gene are known in the art (see, e.g., WO 02/09746). Furthermore, the nucleic acid sequences of genes encoding genomic fHbp variants and subvariants are known, providing for ready adaptation of such methods in the upregulation of endogenous fHbp expression.

The endogenous fHbp may be of any desired variant group (e.g., v.1, v.2, v.3, and the like) or subvariant of fHbp. A “canonical” v.1 fHbp polypeptide of strain MC58 is of particular interest. Also of interest is a subvariant fHbp polypeptide of strain NZ98/294, and v.2 fHbp polypeptide of strain 2996. Also of interest are chimeric fHbp, either those occurring naturally or engineered to contain epitopes that elicit anti-fHbp antibodies that would recognize fHbp from different variant groups. Examples of chimeric fHbp include, e.g., a first component from a fHBP v.1 polypeptide and a second component from a fHBP v.2 polypeptide.

Modification of a Neisserial host cell to provide for increased production of endogenous fHbp may include partial or total replacement of all of a portion of the endogenous gene controlling fHbp expression, where the modification provides for, for example, enhanced transcriptional activity relative to the unmodified parental strain. Increased transcriptional activity may be conferred by variants (point mutations, deletions and/or insertions) of the endogenous control regions, by naturally occurring or modified heterologous promoters or by a combination of both. In general the genetic modification confers a transcriptional activity greater than that of the unmodified endogenous transcriptional activity (e.g., by introduction of a strong promoter), resulting in an enhanced expression of fHbp.

Typical strong promoters that may be useful in increasing fHbp transcription production can include, for example, the promoters of nmb1523, porA, porB, lbpB, tbpB, p110, hpuAB, lgtF, Opa, p110, 1st, and hpuAB. Promoters of porA, Rmp and porB are of particular interest as constitutive, strong promoters. PorB promoter activity is contained in a fragment corresponding to nucleotides −1 to −250 upstream of the initiation codon of porB.

Methods are available in the art to accomplish introduction of a promoter into a host cell genome so as to operably link the promoter to an endogenous fHbp-encoding nucleic acid. For example, double cross-over homologous recombination technology to introduce a promoter in a region upstream of the coding sequence, e.g., about 1000 bp, from about 30-970 bp, about 200-600 bp, about 300-500 bp, or about 400 bp upstream (5′) of the initiation ATG codon of the fHbp-encoding nucleic acid sequence to provide for up-regulation. Optimal placement of the promoter can be determined through routine use of methods available in the art.

For example, a highly active promoter (e.g., PorA, PorB or Rmp promoters) upstream of the targeted gene. As an example, the PorA promoter can be optimized for expression as described by van der Ende et al. Infect Immun 2000; 68:6685-90. Insertion of the promoter can be accomplished by, for example, PCR amplification of the upstream segment of the targeted fHbp gene, cloning the upstream segment in a vector, and either inserting appropriate restriction sites during PCR amplification, or using naturally occurring restriction sites to insert the PorA promoter segment. For example, an about 700 bp upstream segment of the fHbp gene can be cloned. Using naturally occurring restriction enzyme sites located at an appropriate distance (e.g., about 400 bp) upstream of the fHbp promoter within this cloned segment a PorA promoter segment is inserted. An antibiotic (e.g., erythromycin) resistance cassette can be inserted within the segment further upstream of the PorA promoter and the construct was used to replace the wild-type upstream fHbp segment by homologous recombination.

Another approach involves introducing a fHbp polypeptide-encoding sequence downstream of an endogenous promoter that exhibits strong transcriptional activity in the host cell genome. For example, the coding region of the Rmp gene can be replaced with a coding sequence for a fHbp polypeptide. This approach takes advantage of the highly active constitutive Rmp promoter to drive expression.

Neisserial Host Cells Genetically Modified to Express an Exogenous fHbp

Neisserial strains can be genetically modified to over-express fHbp by introduction of a construct encoding a fHbp polypeptide into a Neisserial host cell. The fHbp introduced for expression is referred to herein as an “exogenous” fHbp. The host cell produces an endogenous fHbp, the exogenous fHbp may have the same or different amino acid sequence compared to the endogenous fHbp. In some embodiments, the endogenous fHbp is not modified while in certain other embodiments, the endogenous fHbp is disrupted, for example, knocked out.

The strain used as the host cell in this embodiment can produce any level of fHbp (e.g., high level, intermediate level, or low level fHbp production). For example, a strain that is selected for low level or no detectable fHbp production, or that is modified to exhibit no detectable, or a low level, of fHbp production. For example, the host cell may be genetically modified so that the endogenous fHbp gene is disrupted so that fHbp is not produced or is not present in the cell envelope (and thus is not present at detectable levels in a vesicle prepared from such a modified cell). In other embodiments, the host cell produces an intermediate or high level of fHbp (e.g., relative to a level of fHbp produced by, for example, RM1090).

fHbp Polypeptides

The host cell can be genetically modified to express any suitable fHbp polypeptide, including fHbp variants or subvariants. As described in more detail below, the amino acid sequences of many fHbp polypeptides are known; alignment of these sequences provides guidance as to residues that are conserved among the variants, thus providing guidance as to amino acid modifications (e.g., substitutions, insertions, deletions) that can be made.

Accordingly, “fHbp polypeptide” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring fHbp polypeptide, and which are capable of eliciting antibodies that specifically bind a naturally-occurring fHbp polypeptide present on a whole cell Neisserial bacterium. “fHbp polypeptide” also encompasses fusion proteins, e.g., a fHbp polypeptide having a heterologous polypeptide at the N- and/or C-terminus.

The host cell can be genetically modified to express at least 1 fHbp polypeptide, and can be modified to express 2, 3, 4 or more fHbp polypeptides in the same host cell. For example, a single host cell can be genetically modified to express at least one variant 1 fHbp polypeptide, at least one variant 2 fHbp polypeptide, and at least one variant 3 fHbp polypeptide.

Where expression of multiple fHbp polypeptides meets with difficulty due to toxicity to the host cell, the different fHbp polypeptides may be expressed from different promoters so as to allow a range of expression. For example, varying both the base composition and number of bases between the −10 and −35 regions of the PorA promoter should result in a wide range of expression of the desired recombinant protein (van der Ende et al. Infect Immun 2000; 68:6685-90).

Nucleic acids encoding a fHbp polypeptide for use in the present disclosure are known in the art. Suitable fHbp polypeptides are described in, for example, WO 2004/048404; Masignani et al. 2003 J Exp Med 197:789-799; Fletcher et al. Infect Immun 2004 2088-2100; Welsch et al. J Immunol 2004 172:5606-5615; and WO 99/57280. Nucleic acid (and amino acid sequences) for fHbp variants and subvariants are also provided in GenBank as accession nos.: NC_{—}003112, GeneID: 904318 (NCBI Ref. NP_{—}274866) (from N. meningitidis strain MC58); AY548371 (AAT01290.1) (from N. meningitidis strain CU385); AY548370 (AAT01289.1) (from N. meningitidis strain H44/76); AY548377 (AAS56920.1) (from N. meningitidis strain M4105); AY548376 (AAS56919.1) (from N. strain M1390); AY548375 (AAS56918.1) (from N. meningitidis strain N98/254); AY548374 (AAS56917.1) (from N. meningitidis strain M6190); AY548373 (AAS56916.1) (from N. meningitidis strain 4243); and AY548372 (AAS56915.1) (from N. meningitidis strain BZ83).

The immature fHbp protein includes a leader sequence of about 19 residues, with each variant usually containing an N-terminal cysteine to which a lipid moeity can be covalently attached. This cysteine residue is usually lipidated in the naturally-occurring protein. “1” indicates that first amino acid of the mature protein, with amino acids indicated by negative numbers part of the leader sequence. Exemplary amino acid sequences of fHbp variants 1, 2 and 3 from N. meningitidis (e.g., from strains MC58, 951-5945, and M1239), are described in WO 2004/048404. Additional amino acid sequences of fHbp polypeptides, including non-naturally occurring variants, is available in WO 2006/081259.

The fHbp can be lipidated or non-lipidated. It is generally preferred that the fHbp be lipidated, so as to provide for positioning of the polypeptide in the membrane. Lipidated fHbp can be prepared by expression of the fHbp polypeptide having the N-terminal signal peptide to direct lipidation by diacylglycerol transferase, followed by cleavage by lipoprotein-specific (type II) signal peptidase.

The fHbp polypeptide useful in the present disclosure includes non-naturally occurring (artificial or mutant) fHbp polypeptides that differ in amino acid sequence from a naturally-occurring fHbp polypeptide, but which are present in the membrane of a Nesserial host so that vesicles prepared from the host contain fHbp in a form that provides for presentation of epitopes of interest, preferably a bactericidal epitope, and provides for an anti-fHbp antibody response. In one embodiment, the fHbp polypeptide is a variant 1 (v.1) or variant 2 (v.2) or variant 3 (v.3) fHbp polypeptide, with subvariants of v.1 v.2 and v.3 being of interest, including subvariants of v.1 (see, e.g., Welsch et al. J Immunol 2004 172:5606-5615). In one embodiment, the fHbp polypeptide comprises an amino acid sequence of a fHbp polypeptide that is most prevalent among the strains endemic to the population to be vaccinated.

fHbp polypeptides useful in the present disclosure also include fusion proteins, where the fusion protein comprises a fHbp polypeptide having a fusion partner at its N-terminus or C-terminus. Fusion partners of interest include, for example, glutathione S transferase (GST), maltose binding protein (MBP), His-tag, and the like, as well as leader peptides from other proteins, particularly lipoproteins (e.g., the amino acid sequence prior to the N-terminal cysteine may be replaced with another leader peptide of interest).

Other fHbp polypeptide-encoding nucleic acids can be identified using techniques well known in the art, where fHbp polypeptides can be identified based on amino acid sequences similarity to a known fHbp polypeptide. Such fHbp polypeptides generally share at least about 60%, 65%, 70%, 75%, 80%, 85%, 90%, 95% or greater sequence identity at the nucleotide or amino acid level. Sequence identity can be determined using methods for alignment and comparison of nucleic acid or amino acid sequences, which methods are well known in the art. Comparison of longer sequences may require more sophisticated methods to achieve optimal alignment of two sequences. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85:2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e. resulting in the highest percentage of sequence similarity over the comparison window) generated by the various methods is selected.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. Polypeptides of interest include those having at least 60%, 70%, 75%, 80%, 85%, 90%, 95% or more nucleotide or amino acid residue identity, when compared and aligned for maximum correspondence, as measured using one of the following sequence comparison algorithms or by visual inspection. Preferably, the region sharing sequence identity exists over a region of the sequences that is at least about 10, 20, 30, 40, 50, 60, 70, 80, or 100 contiguous residues in length. In a most preferred embodiment, identity of the sequences is determined by comparison of the sequences over the entire length of the coding region of a reference polypeptide.

For sequence comparison, typically one sequence acts as a reference sequence (e.g., a naturally-occurring fHbp polypeptide sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra).

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides share sequence identity is that the polypeptide encoded by the first nucleic acid is immunologically cross reactive with the polypeptide encoded by the second nucleic acid, as described below. Thus, a polypeptide typically share sequence identity with a second polypeptide, for example, where the two polypeptides differ only by conservative substitutions. Another indication that two nucleic acid sequences share sequence identity is that the two molecules hybridize to each other under stringent conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the present disclosure.

Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Vector and Methods for Introducing Genetic Material into Neisserial Host Cells

Methods and compositions which can be readily adapted to provide for genetic modification of a Neisserial host cell to express an exogenous fHbp polypeptide are known in the art. Exemplary vectors and methods are provided in WO 02/09746 and O'Dwyer et al. Infect Immun 2004; 72:6511-80.

Methods for transfer of genetic material into a Neisserial host include, for example, conjugation, transformation, electroporation, calcium phosphate methods and the like. The method for transfer should provide for stable expression of the introduced fHbp-encoding nucleic acid. The fHbp-encoding nucleic acid can be provided as a inheritable episomal element (e.g., plasmid) or can be genomically integrated.

Suitable vectors will vary in composition depending what type of recombination event is to be performed. Integrative vectors can be conditionally replicative or suicide plasmids, bacteriophages, transposon or linear DNA fragments obtained by restriction hydrolysis or PCR amplification. Selection of the recombination event can be accomplished by means of selectable genetic marker such as genes conferring resistance to antibiotics (for instance kanamycin, erythromycin, chloramphenicol, or gentamycin), genes conferring resistance to heavy metals and/or toxic compounds or genes complementing auxotrophic mutations (for instance pur, leu, met, aro).

In one embodiment, the vector is an expression vector based on episomal plasmids containing selectable drug resistance markers that autonomously replicate in both E. coli and N. meningitidis. One example of such a “shuttle vector” is the plasmid pFP10 (Pagotto et al. Gene 2000 244:13-19). In one embodiment, the vector is pComP1523 containing the strong promoter from gene nmb1523, which allows constitutive expression of the gene of interest (Ieva R et al., J Bacteriol 2005; 187:3421-30).

### Preparation of Neisseria Meningitidis Vesicles

The antigenic compositions for use in the present disclosure generally include vesicles prepared from Neisserial cells genetically modified to provide for decreased or no activity of the product of the lpxL1 gene and that are express an acceptable level of fHbp, either naturally or due to genetic modification (e.g., due to expression of a recombinant fHbp). As referred to herein “vesicles” is meant to encompass outer membrane vesicles as well as microvesicles (which are also referred to as blebs).

In one embodiment, the antigenic composition comprises outer membrane vesicles (OMV) prepared from the outer membrane of a cultured strain of Neisseria meningitidis spp. OMVs may be obtained from a Neisseria meningitidis grown in broth or solid medium culture, preferably by separating the bacterial cells from the culture medium (e.g. by filtration or by a low-speed centrifugation that pellets the cells, or the like), lysing the cells (e.g. by addition of detergent, osmotic shock, sonication, cavitation, homogenization, or the like) and separating an outer membrane fraction from cytoplasmic molecules (e.g. by filtration; or by differential precipitation or aggregation of outer membranes and/or outer membrane vesicles, or by affinity separation methods using ligands that specifically recognize outer membrane molecules; or by a high-speed centrifugation that pellets outer membranes and/or outer membrane vesicles, or the like); outer membrane fractions may be used to produce OMVs.

In another embodiment, the antigenic composition comprises microvesicles (MV) or blebs that are released during culture of said Neisseria meningitidis spp. MVs may be obtained by culturing a strain of Neisseria meningitidis in broth culture medium, separating whole cells from the broth culture medium (e.g. by filtration, or by a low-speed centrifugation that pellets only the cells and not the smaller blebs, or the like), and then collecting the MVs that are present in the cell-free culture medium (e.g. by filtration, differential precipitation or aggregation of MVs, or by a high-speed centrifugation that pellets the blebs, or the like). Strains for use in production of MVs can generally be selected on the basis of the amount of blebs produced in culture (e.g., bacteria can be cultured in a reasonable number to provide for production of blebs suitable for isolation and administration in the methods described herein). An exemplary strain that produces high levels of blebs is described in PCT Publication No. WO 01/34642. In addition to bleb production, strains for use in MV production may also be selected on the basis of NspA production, where strains that produce higher levels of NspA may be preferable (for examples of N. meningitides strains having different NspA production levels, see, e.g., Moe et al. (1999 Infect. Immun. 67: 5664).

In another embodiment, the antigenic composition comprises vesicles from one strain, or from 2, 3, 4, 5 or more strains, which strains may be homologous or heterologous, usually heterologous, to one another with respect to one or both of fHbp or PorA. The strains may be from the same or different capsular groups. In one embodiment, the vesicles are prepared from a strain that expresses 1, 2, 3, 4, 5, 6, 7, 8, 9 or 10 or more fHbp proteins, which may be different variants (v.1, v.2, v.3) or subvariants (e.g., a subvariant of v.1, v.2, or v.3). In another embodiment, the antigenic compositions comprise a mixture of OMVs and MVs, which may be from the same or different strains. In such embodiments, vesicles from different strains may be administered as a mixture. For example, one antigenic composition may comprise vesicles from two or more strains, where at least a first strain, genetically modified to provide for decreased or no activity of the product of the lpxL1 gene, expresses one, two, or more variants of fHbp proteins and the second strain, genetically modified to provide for decreased or no activity of the product of the lpxL1 gene, expresses one variant of fHbp protein. The variants or subvariants expressed by these two strains may be the same or different and may be endogenous or exogenous to the respective strains. In addition to vesicles (OMVs and/or MVs), isolated antigens or particular combinations of antigens (for example, recombinant proteins or recombinant vaccines) may be included in the antigenic compositions of the present disclosure. Exemplary antigens include, but are not limited to, recombinant proteins, such as recombinant fHbp, PorA, and/or NadA.

### Reduction of Lipid Toxicity

Where desired (e.g., where the strains used to produce vesicles are associated with endotoxin or particular high levels of endotoxin), the vesicles are optionally treated to reduce endotoxin, e.g., to reduce toxicity following administration. Although less desirable as discussed below, reduction of endotoxin can be accomplished by extraction with a suitable detergent (for example, BRIJ-96, sodium deoxycholate, sodium lauroylsarcosinate, Empigen BB, Triton X-100, TWEEN 20 (sorbitan monolaurate polyoxyethylene), TWEEN 80, at a concentration of 0.1-10%, preferably 0.5-2%, and SDS). Where detergent extraction is used, it is preferable to use a detergent other than deoxycholate. In some embodiment, vesicles are produced without use of detergent, e.g., without use of deoxycholate or other detergent.

In embodiments of particular interest, the vesicles of the antigenic compositions are prepared without detergent. Although detergent treatment is useful to remove endotoxin activity, it may deplete the native fHbp lipoprotein by extraction during vesicle production. Thus it may be particularly desirable to decrease endotoxin activity using technology that does not require a detergent. In one approach, strains that are relatively low producers of endotoxin (lipopolysaccharide, LPS) are used so as to avoid the need to remove endotoxin from the final preparation prior to use in humans. For example, the vesicles can be prepared from Neisseria mutants in which lipooligosaccharide or other antigens that may be undesirable in a vaccine (e.g. Rmp) is reduced or eliminated.

LPS toxic activity can also be altered by introducing mutations in genes/loci involved in polymyxin B resistance (such resistance has been correlated with addition of aminoarabinose on the 4′ phosphate of lipid A). These genes/loci could be pmrE that encodes a UDP-glucose dehydrogenase, or a region of antimicrobial peptide-resistance genes common to many enterobacteriaciae which could be involved in aminoarabinose synthesis and transfer. The gene pmrF that is present in this region encodes a dolicol-phosphate manosyl transferase (Gunn J. S., Kheng, B. L., Krueger J., Kim K., Guo L., Hackett M., Miller S. I. 1998. Mol. Microbiol. 27: 1171-1182).

Mutations in the PhoP-PhoQ regulatory system, which is a phospho-relay two component regulatory system (e.g., PhoP constitutive phenotype, PhoPc), or low Mg++ environmental or culture conditions (that activate the PhoP-PhoQ regulatory system) lead to the addition of aminoarabinose on the 4′-phosphate and 2-hydroxymyristate replacing myristate (hydroxylation of myristate). This modified lipid A displays reduced ability to stimulate E-selectin expression by human endothelial cells and TNF-α secretion from human monocytes.

Polymyxin B resistant strains are also suitable for use in the present disclosure, as such strains have been shown to have reduced LPS toxicity (see, e.g., van der Ley et al. 1994. In: Proceedings of the ninth international pathogenic Neisseria conference. The Guildhall, Winchester, England). Alternatively, synthetic peptides that mimic the binding activity of polymyxin B may be added to the antigenic compositions to reduce LPS toxic activity (see, e.g., Rustici et al. 1993, Science 259:361-365; Porro et al. Prog Clin Biol Res. 1998; 397:315-25).

Endotoxin can also be reduced through selection of culture conditions. For example, culturing the strain in a growth medium containing 0.1 mg-100 mg of aminoarabinose per liter medium provides for reduced lipid toxicity (see, e.g., WO 02/097646).

In an embodiment of particular interest, vesicles are prepared from N. meningitidis strains that contain genetic modifications that result in decreased or no detectable toxic activity of lipid A. For example, such strain can be genetically modified in lipid A biosynthesis (Steeghs et al. Infect Immun 1999; 67:4988-93; van der Ley et al. Infect Immun 2001; 69:5981-90; Steeghs et al. J Endotoxin Res 2004; 10:113-9). The immunogenic compositions of the present disclosure may be detoxified by modification of LPS, such as downregulation and/or inactivation of the enzymes encoded by lpxL1 or lpxL2, respectively. Production of a penta-acylated lipid A made in lpxL1 mutants indicates that the enzyme encoded by lpxL1 adds the C12 to the N-linked 3-OH C14 at the 2′ position of GlcN II. The major lipid A species found in lpxL2 mutants is tetra-acylated, indicating the enzyme encoded by lpxL2 adds the other C12, i.e., to the N-linked 3-OH C14 at the 2 position of GlcN I. Mutations resulting in a decreased (or no) expression of these genes (or decreased or no activity of the products of these genes) result in altered toxic activity of lipid A (van der Ley et al. 2001; 69:5981-90). Tetra-acylated (lpxL2 mutant) and penta acylated (lpxL1 mutant) lipid A are less toxic than the wild-type lipid A. Mutations in the lipid A 4′-kinase encoding gene (lpxK) also decreases the toxic activity of lipid A.

Of particular interest for use in production of vesicles (e.g., MV or OMV) are N. meningitidis strains genetically modified so as to provide for decreased or no detectable functional LpxL1-encoded protein. Such vesicles provide for reduced toxicity as compared to N. meningitidis strains that are wildtype for LPS production, while retaining immunogenicity of fHbp.

Methods for genetically modifying a locus of interest in N. meningitidis are known in the art, and can be readily employed to generate the mutations discussed above.

### Compositions Comprising Vesicle Vaccines

The present disclosure provides compositions comprising vesicle vaccines, wherein the vesicles are prepared from one, two, three or more strains of Neisseria, which strains may be genetically diverse to one another. Each of the strains used for the production of vesicle vaccines can be genetically modified so as to provide for vesicles having reduced toxicity (e.g., relative to vesicles produced from the same strain that is not so genetically modified).

Genetic modification to decrease endotoxicity of the vaccine may include introducing mutations in genes required for lipid A biosynthesis, where such genetic modification may significantly decrease the expression of the protein encoded by a gene required for lipid A biosynthesis or may completely disrupt the expression of the protein encoded by the gene or may result in the expression of a non-functional protein. In certain embodiments, a knock out technique may be used to disrupt one or more genes required for lipid A biosynthesis. In certain aspects, the disrupted gene is lpxL1, lpxL2, or lpxK. In certain embodiments, one or more of these genes is disrupted. Genetic modification for decreased endotoxicity of vesicle vaccines enables production of non-detergent treated or native vesicle vaccines. Strains having a genetic modification to decrease LpxL1 activity are of particular interest.

In addition to being genetically modified to decrease endotoxicity (e.g., by genetic modification to provide for decreased or no detectable activity of LpxL1), each of the Neisseria bacterium used for the production of vesicle vaccines, may be further genetically modified to provide for expression fHbp, which fHbp may be heterologous to the strain. Where the strain is genetically modified to express an fHbp, the strain may be optionally modified to disrupt production of an endogenous fHbp.

Accordingly, the strains used for production of vesicles vaccines may be genetically modified for decreased or no activity of the product of lpxL1 gene. Such strains may express an endogenous fHbp, express an endogenous fHbp and a recombinant fHbp, or may be genetically modified to disrupt production of endogenous fHbp polypeptide and to express a recombinant fHbp polypeptide, which recombinant fHbp may be heterologous to the host strain or may be of the same variant type as the endogenous fHbp polypeptide. For example, if the endogenous fHbp polypeptide is fHbp v.1, the heterlogous fHbp polypeptide may be fHbp v. 2 or v. 3 or a subvariant of fHbp, such as v.1.10, 1.3. 1.4, 1.2, etc.

Monovalent vaccines can be produced from a single Neisserial strain genetically modified to decrease endotoxicity, and which may be further modified as described above. Parent strains for use in preparation of such monovalent vaccines include, but are not limited to, RM1090, H44/76 and NZ98/254. In certain embodiments, a monovalent vaccine is combined with isolated Neisserial proteins. Exemplary Neisserial proteins include, but are not limited to, fHbp, PorA, and/or NadA. In certain embodiments, a monovalent vaccine is combined with recombinant 5C vaccine. Recombinant 5C vaccine or r5CV refers to a recombinant protein vaccine containing GNA2091 fused with fHbp v. 1, GNA2132 (from NZ98/254) fused with GNA1030, and NadA (Giuliani Giuliani et al. Proc Natl Acad Sci USA 2006; 103:10834-9). In certain other embodiments, a monovalent vaccine is combined with recombinant 3C vaccine. Recombinant 3C protein vaccine or r3C refers to a vaccine containing recombinant fHbp v.1 (fHbp encoded by gene from strain MC58), GNA2132 (encoded by gene from strain NZ98/254), and NadA (encoded by the gene from strain 2996).

The present disclosure also provides bivalent vaccines, which contain vesicles from two Neisserial strains genetically modified to decrease endotoxicity, where the two strains are genetically diverse to one another. For example, bivalent vaccines can be prepared from strains that are classified into different serogroups of serotypes or serosubtypes. The two strains used for production of a bivalent vaccine may be selected such that the two strains are homologous or heterologous, usually heterologous, to one another with respect to one or both of fHbp or PorA. In one embodiment, the bivalent vaccines are prepared from strains expressing different fHbp proteins, which fHbp proteins may be different variants (v.1, v.2, v.3) or subvariants (e.g., a subvariant of v.1, v.2, or v.3). In another embodiment, the bivalent vaccines are prepared from strains that are heterologous to one another respect to PorA. In certain aspects, the bivalent vaccines are prepared from strains that are heterologous to one another respect to fHbp proteins and PorA.

As an example, a bivalent vaccine may be produced from vesicles obtained from a first strain and a second strain, each strain genetically modified to provide for decreased or no activity of the product of the lpxL1 gene. The first and second strains may express endogenous fHbp, or one or both may be genetically modified to express a recombinant fHbp. Optionally, one or both strains may be genetically modified to disrupt production of an endogenous fHbp polypeptide and to express a recombinant fHbp.

For example, a bivalent vaccine may be produced from vesicles obtained from a genetically modified version of the strains H44/76 and NZ98/254, where each strain is genetically modified to provide for decreased or no activity of the product of the lpxL1 gene. In one embodiment, the H44/76 strain is further genetically modified to disrupt production of endogenous fHbp v.1 polypeptide and to express a fHbp polypeptide (e.g., a fHbp v.1 polypeptide) and where the NZ98/254 strain is further genetically modified to express a heterologous fHbp polypeptide, e.g., fHbp v.2.

The compositions of the present disclosure may be produced by combining vesicles with Neisserial antigens. In some embodiments, compositions of the present disclosure may be produced by mixing vesicles from two strains with one or more Neisserial antigen(s) (e.g., with one or more antigens of the r3C or r5C vaccines). Following combination of the vesicles (with or without Neisserial antigens), the vesicles may optionally be adsorbed with an aluminum salt, e.g., aluminum hydroxide, aluminum phosphate. Alternatively, adsorption can be carried out with the vesicles prior to combining with one another and/or with any Neisserial antigens.

Polypeptides Comprising Neisserial Antigens for Combination with Vesicle Vaccine

The compositions of the present disclosure may be produced by combining vesicles with Neisserial antigens. In some embodiments, vesicles are combined with one, two, three, four, five, or more Neisserial antigens, particularly Neisserial polypeptide antigens. “Neisserial antigen” as used herein refers to an antigen, usually a polypeptide, that when administered to a mammal as an immunogenic composition provides for production of antibodies that specifically bind a Neisserial bacterium. “Neisserial antigen” is not meant to limit the antigen to a method of production, and thus does not require the antigen be isolated directly from a Neisseria bacterium. “Neisserial antigen” thus encompasses antigens as isolated from a Neisserial bacterium, recombinant full-length polypeptides having an amino acid sequence of a Neisserial protein of interest (e.g., fHbp, NAD, and the like), as well as antigenic fragments, fusion proteins, and the like, which can be produced from a genetically modified host cell. Where the composition includes more than one Neisserial antigen, such may be derived from one or more strains of Neisseria meningitides, which strains may be genetically diverse.

Polypeptides useful for combining with vesicles to produce compositions of the present disclosure may be one or more full length Neisserial antigens, fragments thereof, fusion polypeptides of two or more full length polypeptides or of fragments thereof, or a combination of full length and fusion polypeptides. Polypeptides useful for combining with vesicle vaccines may be modified, for example, by addition of a heterologous polypeptide sequence, such a purification tag, a stabilizing polypeptide, by addition of sugar residues or lipid residues etc. Methods for purification of wild type polypeptides from cells are well known in the art. Such methods include, for example, use of an affinity column with antibodies that specifically bind to the polypeptide or use of size fractionation columns etc. Methods for purification of recombinant polypeptides from cells are well known in the art. Such methods include, for example, use of affinity column of antibodies that specifically bind to the polypeptide, use of size fractionation columns, or affinity purification of polypeptides with a tag etc.

Any of the polypeptides comprising Neisserial antigens disclosed herein and those known in the art may be used in combination with the vesicles disclosed herein. Examples of polypeptides comprising Neisserial antigens include, for example, fHbp, GNA2132, Nad A, GNA2091 and GNA1030. fHbp may be fHbp v.1, v.2 or v. 3, or a subvariant of a particular variant, such as 1.1, 1.10, 1.2. 1.3 etc. The fHbp variant polypeptide used in the compositions of the present disclosure may be of the same or a different variant group compared to fHbp polypeptide expressed by Neisseria bacterium used to produce the vesicles. Any combination of fHbp, GNA2132, Nad A, GNA2091 and GNA1030 Neisserial antigens are contemplated. Thus a composition may comprise one or more of these Neisserial antigens in addition to the vesicles. In some embodiments two or more of these Neisserial antigens may be expressed as a fusion protein. Some specific combinations include fHbp, GNA2132 and Nad A; and fHbp, GNA2132, Nad A, GNA2091 and GNA1030. In certain embodiments vesicles may be combined with the 5 component recombinant protein vaccine (5C or r5CV). The 5 component recombinant protein vaccine (rc5) is described in Giuliani et al. (Proc Natl Acad Sci USA 2006; 103:10834-9).

### Formulations

Immunogenic compositions used as vaccines comprise an immunologically effective amount of antigen, particularly an immunologically effective amount of fHbp, as well as any other compatible components, as needed. By “immunologically effective amount” is meant that the administration of that amount to an individual, either in a single dose or as part of a series, is effective to elicit for treatment or prevention. This amount varies depending upon the health and physical condition of the individual to be treated, age, the taxonomic group of the individual to be treated (e.g., non-human primate, primate, human, etc.), the capacity of the individual's immune system to synthesize antibodies, the degree of protection desired, the formulation of the vaccine, the treating clinician's assessment of the medical situation, and other relevant factors. It is expected that the amount will fall in a relatively broad range that can be determined through routine trials.

Dosage regimen may be a single dose schedule or a multiple dose schedule (e.g., including booster doses) with a unit dosage form of the antigenic composition administered at different times. The term “unit dosage form,” as used herein, refers to physically discrete units suitable as unitary dosages for human and animal subjects, each unit containing a predetermined quantity of the antigenic compositions of the present disclosure in an amount sufficient to produce the desired effect, which compositions are provided in association with a pharmaceutically acceptable excipient (e.g., pharmaceutically acceptable diluent, carrier or vehicle). The vaccine may be administered in conjunction with other immunoregulatory agents.

The antigenic compositions to be administered are provided in a pharmaceutically acceptable diluent such as an aqueous solution, often a saline solution, a semi-solid form (e.g., gel), or in powder form. Such diluents can be inert, although the compositions of the disclosure may also include an adjuvant. Examples of known suitable adjuvants that can be used in humans include, but are not necessarily limited to, alum, aluminum phosphate, aluminum hydroxide, MF59 (4.3% w/v squalene, 0.5% w/v Tween 80, 0.5% w/v Span 85), CpG-containing nucleic acid (where the cytosine is unmethylated), QS21, MPL, 3DMPL, extracts from Aquilla, ISCOMS, LT/CT mutants, poly(D,L-lactide-co-glycolide) (PLG) microparticles, Quil A, interleukins, and the like. For experimental animals, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogenic antigen.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (W090/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21 or STIMULON™ (Cambridge Bioscience, Worcester, Mass.) may be used or particles generated therefrom such as ISCOMs (immunostimulating complexes), which ISCOMS may be devoid of additional detergent e.g. WO00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO00/56358; (6) combinations of 3dMPL with, for example, QS21 and/oroil-in-water emulsions e.g. EP-A-0835318, EP-A-0735898, EP-A-0761231; (7) oligonucleotides comprising CpG motifs (Krieg Vaccine 2000, 19, 618-622; Krieg Curr opin Mol Ther2001 3:15-24; Roman et al., Nat. Med., 1997, 3, 849-854; Weiner et al., PNAS USA, 1997, 94, 10833-10837; Davis et al, J. Immunol, 1998, 160, 870-876; Chu et at., J. Exp. Med, 1997, 186, 1623-1631; Lipford et al, Ear. J. Immunol., 1997, 27, 2340-2344; Moldoveami e/al., Vaccine, 1988, 16, 1216-1224, Krieg et al., Nature, 1995, 374, 546-549; Klinman et al., PNAS USA, 1996, 93, 2879-2883; Ballas et al, J. Immunol, 1996, 157, 1840-1845; Cowdery et al, J. Immunol, 1996, 156, 4570-4575; Halpern et al, Cell Immunol, 1996, 167, 72-78; Yamamoto et al, Jpn. J. Cancer Res., 1988, 79, 866-873; Stacey et al, J. Immunol., 1996, 157, 2116-2122; Messina et al, J. Immunol, 1991, 147, 1759-1764; Yi et al, J. Immunol, 1996, 157, 4918-4925; Yi et al, J. Immunol, 1996, 157, 5394-5402; Yi et al, J. Immunol, 1998, 160, 4755-4761; and Yi et al, J. Immunol, 1998, 160, 5898-5906; International patent applications WO96/02555, WO98/16247, WO98/18810, WO98/40100, WO98/55495, WO98/37919 and WO98/52581) i.e. containing at least one CG dinucleotide, where the cytosine is unmethylated; (8) a polyoxyethylene ether or a polyoxyethylene ester e.g. WO99/52549; (9) a polyoxyethylene sorbitan ester surfactant in combination with an octoxynol (WO01/21207) or a polyoxyethylene alkyl ether or ester surfactant in combination with at least one additional non-ionic surfactant such as an octoxynol (WO01/21152); (10) a saponin and an immunostimulatory oligonucleotide (e.g. a CpG oligonucleotide) (WO00/62800); (11) an immunostimulant and a particle of metal salt e.g. WO00/23105; (12) a saponin and an oil-in-water emulsion e.g. WO99/11241; (13) a saponin (e.g. QS21)+3dMPL+IM2 (optionally+a sterol) e.g. WO98/57659; (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

The antigenic compositions may be combined with a conventional pharmaceutically acceptable excipient, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antigen in these formulations can vary widely, and will be selected primarily based on fluid volumes, viscosities, body weight and the like in accordance with the particular mode of administration selected and the patient's needs. The resulting compositions may be in the form of a solution, suspension, tablet, pill, capsule, powder, gel, cream, lotion, ointment, aerosol or the like.

The protein concentration of antigenic compositions of the disclosure in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

### Immunization

In general, the methods disclosed herein provide for administration of one or more antigenic compositions of the disclosure to a mammalian subject (e.g., a human) so as to elicit an immune response, particularly a protective immune response, against more than one strain of Neisseria bacteria, and thus protection against disease caused by such bacteria. In particular, the methods of the present disclosure can provide for an immunoprotective immune response against a 1, 2, 3, 4, or more strains of Neisseria meningitidis, where the strains differ in at least one of serogroup, serotype, serosubtype, or fHbp polypeptide (e.g., different fHbp variants and/or subvariants). Of particular interest is induction of a protective immune response against multiple strains of Neisseria meningitidis of serogroup B, particularly where the strains differ in serosubtype (e.g., have heterologous PorAs). Also of particular interest is induction of a protective immune response against strains that are heterologous to one other in terms of PorA and/or fHbp.

The antigenic compositions of the disclosure can be administered orally, nasally, nasopharyngeally, parenterally, enterically, gastrically, topically, transdermally, subcutaneously, intramuscularly, in tablet, solid, powdered, liquid, aerosol form, locally or systemically, with or without added excipients. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

It is recognized that oral administration can require protection of the compositions from digestion. This is typically accomplished either by association of the composition with an agent that renders it resistant to acidic and enzymatic hydrolysis or by packaging the composition in an appropriately resistant carrier. Means of protecting from digestion are well known in the art.

The compositions are administered to an animal that is at risk from acquiring a Neisserial disease to prevent or at least partially arrest the development of disease and its complications. An amount adequate to accomplish this is defined as a “therapeutically effective dose.” Amounts effective for therapeutic use will depend on, e.g., the antigenic composition, the manner of administration, the weight and general state of health of the patient, and the judgment of the prescribing physician. Single or multiple doses of the antigenic compositions may be administered depending on the dosage and frequency required and tolerated by the patient, and route of administration.

The antigenic compositions described herein can comprise a mixture of vesicles (e.g., OMV and MV), which vesicles can be from the same or different strains. In another embodiment, the antigenic compositions can comprise a mixture of vesicles from 2, 3, 4, 5 or more strains, where the vesicles can be OMV, MV or both.

The antigenic compositions are administered in an amount effective to elicit an immune response, particularly a humoral immune response, in the host. Amounts for the immunization of the mixture generally range from about 0.001 mg to about 1.0 mg per 70 kilogram patient, more commonly from about 0.001 mg to about 0.2 mg per 70 kilogram patient. Dosages from 0.001 up to about 10 mg per patient per day may be used, particularly when the antigen is administered to a secluded site and not into the blood stream, such as into a body cavity or into a lumen of an organ. Substantially higher dosages (e.g. 10 to 100 mg or more) are possible in oral, nasal, or topical administration. The initial administration of the mixture can be followed by booster immunization of the same of different mixture, with at least one booster, more usually two boosters, being preferred.

In one embodiment, the antigenic compositions used to prime and boost are prepared from strains of Neisseria that possess variant immunodominant antigens (the main antigens that are routinely detected by antisera from different host animals that have been infected with Neisseria; representative examples include Porin A, Porin B, pilin, NspA, phospholipids, polysaccharides, lipopolysaccharides, pilins, OmpA, Opa, Opc, etc.) and/or variant fHbp proteins. The strains also may vary with respect to the capsule molecule, as reflected by their serogroup.

Serotype and serosubtype classification is currently determined by detecting which of a panel of known monoclonals, which are known to recognize specific Porin molecules, bind to an unknown strain (Sacchi et al., 1998, Clin. Diag. Lab. Immunol. 5:348). It is probable that other such monoclonals will be identified. The use of any novel serotypes and serosubtypes which may be defined by any new monoclonals are specifically contemplated by the present disclosure. In addition, serotypes and serosubtypes may be defined, not only by interaction with monoclonal antibodies, but also structurally by the absence and/or presence of defined peptide residues and peptide epitopes (Sacchi et al., 2000, J. Infect. Dis. 182:1169). Serotype and serosubtype classification schemes that are based on structural features of the Porins (known or that may be discovered at a later date) are specifically encompassed by the present disclosure.

In another embodiment, the antigenic compositions administered are prepared from 2, 3, 4, 5 or more strains, which strains may be homologous or heterologous, usually heterologous, to one another with respect to one or both of fHbp or PorA. In one embodiment, the vesicles are prepared from strains express different fHbp proteins, which fHbp proteins may be different variants (v.1, v.2, v.3) or subvariants (e.g., a subvariant of v.1, v.2, or v.3). In another embodiment, the vesicles are prepared from strains that are heterologous to one another respect to PorA.

In embodiments of particular interest, vesicles are prepared from Neisserial strains that are genetically diverse to one another (e.g., the strains belong to different serotypes and/or serosubtypes; express different PorA proteins; express different fHbp variants or subvariants; and/or may also optionally belong to different capsular serogroups). The vesicles can be used to prepare an antigenic composition that is a mixture of vesicles prepared from at least 2, 3, 4, or more of such genetically diverse strains. For example, fHbp protein and/or PorA of the second Neisserial strain from which antigenic compositions are prepared and administered is/are different from that of the first strain used to produce vesicles.

The second, third, and further administered antigenic compositions can optionally be prepared from Neisserial strains are genetically diverse to the second strain (e.g., the strains belong to different serotypes and/or serosubtypes; express different fHbp proteins; express different PorA proteins; and/or belong to different capsular serogroups). For example, a third strain used for preparing a third antigenic composition may be genetically diverse to the first and second strains used to prepare the first and second antigenic compositions, but may, in some embodiments, not be genetically diverse with respect to the first strain.

The present disclosure also contemplates that the antigenic compositions may be obtained from one or more strains of Neisseria, particularly Neisseria meningitidis, that are genetically engineered by known methods (see, e.g. U.S. Pat. No. 6,013,267) to express one or more nucleic acids that encode fHbp. The host cell may express an endogenous fHbp polypeptide or may be modified or selected so as not to express any detectable endogenous fHbp polypeptide. The fHbp polypeptide expressed in the host cell by recombinant techniques (i.e., the exogenous fHbp polypeptide) can be of the same or different variant type as an endogenous fHbp polypeptide.

The host cells may be further modified to express additional antigens of interest, such as Porin A, Porin B, NspA, pilin, or other Neisserial proteins. In addition, the antigen compositions of the disclosure can comprise additional Neisserial antigens such as those exemplified in PCT Publication Nos. WO 99/24578, WO 99/36544; WO 99/57280, WO 00/22430, and WO 00/66791, as well as antigenic fragments of such proteins.

The antigen compositions are typically administered to a mammal that is immunologically naïve with respect to Neisseria, particularly with respect to Neisseria meningitidis. In a particular embodiment, the mammal is a human child about five years or younger, and preferably about two years old or younger, and the antigen compositions are administered at any one or more of the following times: two weeks, one month, 2, 3, 4, 5, 6, 7, 8, 9, 10, or 11 months, or one year or 15, 18, or 21 months after birth, or at 2, 3, 4, or 5 years of age.

In general, administration to any mammal is preferably initiated prior to the first sign of disease symptoms, or at the first sign of possible or actual exposure to Neisseria.

### Passive Immunity

The present disclosure also contemplates immunoprotective antibodies generated by immunization with an antigenic composition of the disclosure, and methods of use. Such antibodies can be administered to an individual (e.g., a human patient) to provide for passive immunity against a Neisserial disease, either to prevent infection or disease from occurring, or as a therapy to improve the clinical outcome in patients with established disease (e.g. decreased complication rate such as shock, decreased mortality rate, or decreased morbidity, such as deafness).

Antibodies administered to a subject that is of a strain other than the strain in which they are raised are often immunogenic. Thus, for example, murine or porcine antibodies administered to a human often induce an immunologic response against the antibody. The immunogenic properties of the antibody are reduced by altering portions, or all, of the antibody into characteristically human sequences thereby producing chimeric or human antibodies, respectively.

Chimeric antibodies are immunoglobulin molecules comprising a human and non-human portion. More specifically, the antigen combining region (or variable region) of a humanized chimeric antibody is derived from a non-human source (e.g. murine), and the constant region of the chimeric antibody (which confers biological effector function to the immunoglobulin) is derived from a human source. The chimeric antibody should have the antigen binding specificity of the non-human antibody molecule and the effector function conferred by the human antibody molecule. A large number of methods of generating chimeric antibodies are well known to those of skill in the art (see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431 and 4,975,369). An alternative approach is the generation of humanized antibodies by linking the CDR regions of non-human antibodies to human constant regions by recombinant DNA techniques. See Queen et al., Proc. Natl. Acad. Sci. USA 86: 10029-10033 (1989) and WO 90/07861.

In one embodiment, recombinant DNA vector is used to transfect a cell line that produces an antibody against a peptide of the disclosure. The novel recombinant DNA vector contains a “replacement gene” to replace all or a portion of the gene encoding the immunoglobulin constant region in the cell line (e.g. a replacement gene may encode all or a portion of a constant region of a human immunoglobulin, or a specific immunoglobulin class), and a “target sequence” which allows for targeted homologous recombination with immunoglobulin sequences within the antibody producing cell.

In another embodiment, a recombinant DNA vector is used to transfect a cell line that produces an antibody having a desired effector function (e.g. a constant region of a human immunoglobulin), in which case, the replacement gene contained in the recombinant vector may encode all or a portion of a region of an antibody and the target sequence contained in the recombinant vector allows for homologous recombination and targeted gene modification within the antibody producing cell. In either embodiment, when only a portion of the variable or constant region is replaced, the resulting chimeric antibody may define the same antigen and/or have the same effector function yet be altered or improved so that the chimeric antibody may demonstrate a greater antigen specificity, greater affinity binding constant, increased effector function, or increased secretion and production by the transfected antibody producing cell line, etc.

In another embodiment, this disclosure provides for fully human antibodies. Human antibodies consist entirely of characteristically human polypeptide sequences. The human antibodies of this disclosure can be produced by a wide variety of methods (see, e.g., Larrick et al., U.S. Pat. No. 5,001,065). In one embodiment, the human antibodies of the present disclosure are produced initially in trioma cells (descended from three cells, two human and one mouse). Genes encoding the antibodies are then cloned and expressed in other cells, particularly non-human mammalian cells. The general approach for producing human antibodies by trioma technology has been described by Ostberg et al. (1983), Hybridoma 2: 361-367, Ostberg, U.S. Pat. No. 4,634,664, and Engelman et al., U.S. Pat. No. 4,634,666. Triomas have been found to produce antibody more stably than ordinary hybridomas made from human cells.

Methods for producing and formulation antibodies suitable for administration to a subject (e.g., a human subject) are well known in the art. For example, antibodies can be provided in a pharmaceutical composition comprising an effective amount of an antibody and a pharmaceutical excipients (e.g., saline). The pharmaceutical composition may optionally include other additives (e.g., buffers, stabilizers, preservatives, and the like). An effective amount of antibody is generally an amount effective to provide for protection against Neisserial disease or symptoms for a desired period, e.g., a period of at least about 2 days to 10 days or 1 month to 2 months).

### Diagnostic Assays

The antigenic compositions of the disclosure, or antibodies produced by administration of such compositions, can also be used for diagnostic purposes. For instance, the antigenic compositions can be used to screen pre-immune and immune sera to ensure that the vaccination has been effective. Antibodies can also be used in immunoassays to detect the presence of particular antigen molecules associated with Neisserial disease.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Materials and Methods

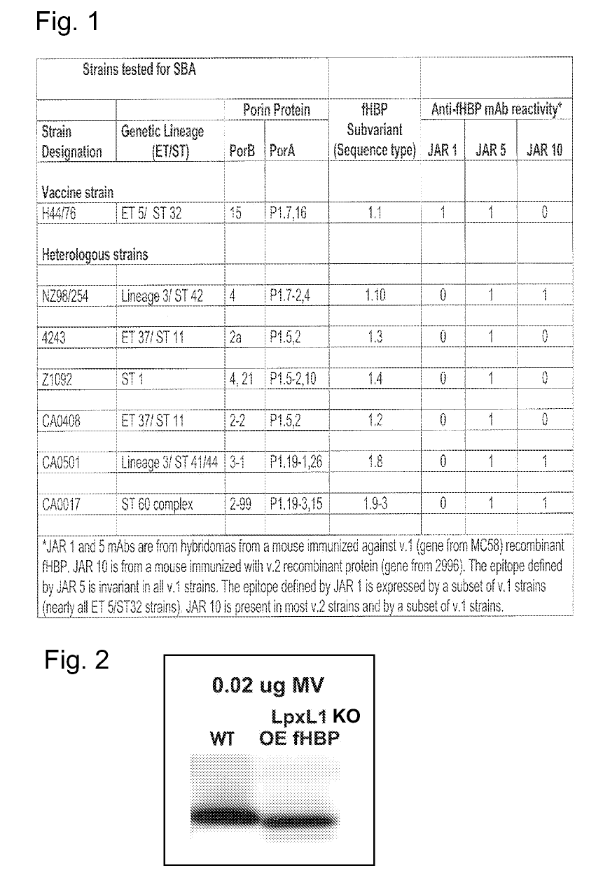

Neisserial strains. The strains used for preparing mutants or testing serum bactericidal activity in Examples 1 and 2 (native MV vaccine prepared from LpxL1 KO mutant of strain H44/76 with endogenous fHbp inactivated and expressing fHbp v.1 from a heterologous promoter) are shown in FIG. 1. These strains are genetically diverse as determined by different sequence type complexes and express fHbp in the v.1 group. Six of the seven strains express PorA heterologous to that of the H44/76 strain used to prepare the MV vaccines. The strains used for preparing MV vaccines or testing serum bactericidal activity in Example 3 (native MV 2 vaccine, prepared from LpxL1 KO mutant of strain NZ98/254 with endogenous fHbp v.1 and expressing a heterologous fHbp v.2, given alone, or in combination with the H44/76 MV 1 vaccine described above, or with three recombinant proteins (fHbp v.1, GNA2132 and NadA)) are shown in Table 3. These strains are genetically diverse, as defined by multilocus sequencing type, and they also express several different PorA VR sequence types. The strains used for testing serum bactericidal activity in Example 4 are listed in Table 4.

LpxL1 knock-out (KO) mutants. To generate the LpxL1 KO mutant, the LpxL1 gene from strain MC58 was amplified by PCR using primers LpxL1_for HindIII 5′-CCCAAGCTTATCCTTCGGGGATGCAGGTC-3′ and LpxL1_revXbaI: 5′-gctctagagccgtctgaacgtagtcagtaaaaatcggggc-3′. The lpxL1 fragment was cloned into HindIII and XbaI digested plasmid pUC18 resulting in plasmid pUCLpxL1. An internal 204 base pair fragment of the LpxL1 gene was deleted by inverse PCR with plasmid pUCLpxL1 as template using primers LpxL1_del1: 5′-aactgcagcggtgaagtgcggatacagg-3′ and LpxL1_del2: 5′-acgcgtcgacaggatttcggacgcaacg-3′. A kanamycin resistant cassette was ligated with the product from the inverse PCR reaction resulting in plasmid pUCLpxLlkan. pFP12-fHbp shuttle vector construct. Over-expression of fHbp in N. meningitidis strain H44/76, in which the gene encoding endogenous fHbp was inactivated, was accomplished using the shuttle vector FP12, which has an origin of replication from a naturally-occurring plasmid in N. gonorrhoeae and has been shown to transform E. coli and N. meningitidis stably (Pagotto et al. Gene 2000; 244: 13-9). The variant 1 fHbp gene, including the putative FUR box promoter from N. meningitidis strain MC58, was amplified from genomic DNA by PCR using the following primers: fHbp FURSphIF 5′, 5,″-ATCGGCATGCGCCGTTCGGACGACATTTG-3″ and fHbp FURStuIR 3′ 5″-AAGAAGGCCTTTATTGCTTGGCGGCAAGGC-3″. The PCR product was then digested with SphI and Stul restriction endonucleases and ligated into pFP12 plasmid digested with SphI and Stul, which removed the GFP gene. The resulting plasmid, pFP12fHbp, was transformed and propagated in E. coli strain TOP10 competent cells (Invitrogen), which was grown in Luria-Bertani medium at 37° C. under chloramphenicol selection (50 μg/ml).

pComP1523 construct. To engineer NZ98/254 to express fHbp v.2, a similar approach to that followed for expressing fHbp v.1 (described above) was used except that a different plasmid (pComP1523) was used. pComP1523 integrates into the chromosome between nmb1428 and nmb1429 and allows expression of fHbp under control of the strong promoter from nmb1523 (Ieva R et al., J Bacteriol 2005; 187:3421-30). Strain NZ98/254 was transformed with pComP1523 containing the full-length gene of fHbp v.2 from strain 8047 (average of 85% amino acid identity with fHbp v.3). The endogenous fHbp gene was not inactivated, which permitted co-expression of both the endogenous fHbp (subvariant of fHbp v.1) and the heterologous fHbp v.2. The transformation was performed as previously described (Koeberling O. et al., Vaccine 2007; 25 (10), 1912-20), and transformants were selected on GC agar plates containing 5 μg/ml chloramphenicol.

Membrane preparations. MVs were were obtained from blebs released by the bacteria into the supernatant as described in (Moe G. R. et al, Infect Immun 2002; 70: 6021-31); see also WO 02/09643.

Recombinant proteins. The recombinant protein vaccine was expressed in E. coli as previously described using a fHbp DNA sequence encoding six COOH-terminal histidines (His tag) and devoid of the N-terminal sequence coding for the putative leader peptide (Welsch et al. J Immunol 2004; 172:5606-15).

Characterization of vaccines. The protein concentrations were determined by the DC protein assay (Bio-Rad, Richmond, Calif.) and the BCA Protein Assay Kit (Pierce, Rockford, Ill.). The MV preparations were analyzed by 15% SDS-PAGE (12.5% SDS-PAGE for the H44/76 preparations) as described by Laemmli (Nature 1970; 227: 680-5) employing a Mini-Protean II electrophoresis apparatus (Bio-Rad), and Western blot. Samples were suspended in sample buffer (0.06 M Tris.HCl, pH 6.8, 10% (v/v) glycerol, 2% (w/v) SDS, 5% (v/v) 2-mercaptoethanol, 10 μg/ml bromophenol blue) and heated to 100° C. for 5 min. before loading directly onto the gel.

For Western blots, the gel was equilibrated with buffer (48 mM Tris.HCl, 39 mM glycine [pH 9.0] 20% (v/v) methanol) and transferred to a nitrocellulose membrane (Bio-Rad) using a Trans-Blot™ (Bio-Rad) semi-dry electrophoretic transfer cell. The nitrocellulose membranes were blocked with 2% (w/v) non-fat milk in PBS, and reacted with a 1:20,000 dilution of anti-rfHbp-antiserum in PBS containing 1% (w/v) BSA and 1% (w/v) Tween-20. Bound antibody was detected using rabbit anti-mouse IgG+A+M-horseradish peroxidase conjugated polyclonal antibody (Zymed, South San Francisco, Calif.) and “WESTERN LIGHTNING™” chemiluminescence reagents (PerkinElmer Life Sciences, Inc., Boston, Mass.). The detecting anti-fHbp antiserum was from mice immunized sequentially with one injection each of 10 μg of recombinant fHbp v.1 (gene from N. meningitidis strain MC58), followed by a dose of recombinant v.3 protein (gene from strain M1239), and followed by a dose of recombinant v.2 protein (gene from strain 2996). Each injection was separated by 3- to 4-weeks.

Cytokine stimulation assay. Blood was obtained from healthy adult donors and the buffy coats were fractionated by Ficoll density centrifugation. The peripheral blood mononuclear cell (PBMC) layer was recovered, washed three times with RPMI 1640 medium and resuspended in complete medium (RPMI 1640+L-glutamine+25 mM HEPES, containing 10% FCS (HyClone, Logan Utah) and 1% penicillin/streptomycin/glutamine). PBMCs were cultured in 96-well flat bottom plates at a density of 4×105 cells per well. Serial 10-fold dilutions (1 μg/ml to 0.00001 μg/ml final concentration) of native MV prepared from the H44/76 wildtype, or the LpxL1 KO mutant with over-expressed fHbp, or detergent-treated wildtype MV were added. The samples were incubated for 4 hours at 37° C. Cytokine secretion was measured by Bio-Plex analysis (BioRad, Hercules Calif.) according to manufacturer's instructions using the human 27-plex panel. The following soluble proteins were assayed: IL-1β, IL-1ra, IL-2, IL-4, IL-5, IL-6, IL-7, IL-8, IL-9, IL-10, IL-12p70, IL-13, IL-15, IL-17, eotaxin, basic FGF, G-CSF, GM-CSF, IFN-γ, IP-10, MCP-1, MIP-1α, MIP-1β, PDGF-BB, RANTES, TNF-α and VEGF. The concentrations of cytokines were analysed using the Bioplex System and Bioplex Manager 4.1 software.

Immunization. The microvesicles or recombinant fHbp protein were adsorbed with aluminum hydroxide. The recombinant polypeptide antigens were added to a solution containing aluminum hydroxide in water to a final concentration of 300 μg/ml. The MVs were added to a solution containing aluminum hydroxide in water to a final concentration of 10 μg/ml. Histidine and NaCl were added to these solutions to a final concentration of 10 mM histidine and 9 mg/ml NaCl. MV or each recombinant polypeptide was adsorbed to aluminum hydroxide separately and then combined. To produce a composition with MVs from one strain and three recombinant proteins, each individual solution contained a concentration four times higher than the final concentration of the vesicles or the polypeptide. Equal volumes of each of the individual solutions were mixed and injected. For compositions with two different vesicles (vesicles from two different strains), the concentrations of the individual vesicle solutions were four times higher than the final concentration. Equal volumes of the individual vesicle solutions were mixed and then a volume of aluminum hydroxide which is equal to the volume of the two combined solutions was added.

Groups of 4-6 weeks old female CD-1 mice were immunized intraperitoneally (10 to 15 mice per group). As described below, for each injection, the mice received a dose of 2 to 5 μg protein of native or detergent-extracted MV vaccine, which were absorbed with 600 μg of aluminum hydroxide. The total dose of the recombinant protein vaccine was 60 μg (20 μg of each protein), which was absorbed with 600 μg of aluminum hydroxide. Three injections of vaccine were given, separated by three weeks. Blood was collected three weeks after the third injection. Sera were separated by centrifugation and stored frozen.

For Examples 1 and 2, the immunogenicity of a native MV vaccine (produced from vesicles from the LpxL1KO mutant of H44/76 with endogenous fHbp inactivated and expressing an exogenous fHbp v.1 from a heterologous promoter) was investigated in mice using a dose of 2 μg of protein of the vaccine. As controls, additional groups of mice were immunized with a detergent-treated OMV prepared from the wildtype strain of H44/76 (2 μg of protein per dose; vaccine produced by the Norwegian Institute of Public Health, and previously tested in humans), a multicomponent recombinant protein vaccine (three recombinant proteins (20 μg of each; or total dose of 60 μg), which contained five antigens consisting of two fusion proteins (GNA2091-fHbp v.1, GNA2132-GNA1030) and NadA (Giuliani et al. Proc Natl Acad Sci USA 2006; 103:10834-9). An additional group of mice was immunized with aluminum hydroxide alone (600 μg, which was the total dose used to adsorb each of the test vaccines).

For Example 3, a native MV vaccine from the LpxL1 KO mutant of NZ98/254 that expressed both the endogenous subvariant of fHbp v.1 and a heterologous fHbp v.2 was used. The immunogenicity in mice given the monovalent vesicle vaccine alone (NZ98/254 LpxL1 KO, endogenous subvariant of fHbp v.1, heterologous fHpb v.2; dose of 2.5 μg of protein), or given in combination with the native H44/76 LpxL1 KO, fbp KO, expressing fHbp v.1 from a heterologous promoter vesicle vaccine (“bivalent native MV vaccine”, 2.5 μg of each MV, or total dose of 5 μg), or given in combination with three recombinant proteins fHbp v.1 (gene from strain MC58), GNA2132 (gene from strain NZ98/254) and NadA (gene from strain 2996). This combination vaccine is referred to as combination monovalent vaccine (2.5 μg)+recombinant 3C vaccine with a dose of 20 μg of each recombinant protein, or a total dose 60 μg of recombinant proteins.

Absorption of anti-fHbp antibodies. To test the contribution of anti-fHbp antibodies to antibody functional activity, we absorbed serum pools to remove anti-fHbp antibodies. In brief, 100 μl of serum pools diluted 1:2 in PBS buffer containing 10 mM imidazole was added to a column that contained 250 μl of Ni-NTA Sepharose (Qiagen, Valencia, Calif.) that had been complexed with 200 μg of recombinant fHbp-HisTag protein or, as a negative control, recombinant NadA-HisTag protein (Comanducci et al. J Exp Med 2002; 195:1445-54; Hou V. C. et al., J Infect Dis 2005; 192: 580-90). The columns were incubated overnight at 4° C., and washed with 500 μl of PBS buffer containing 10 mM imidazole. Five fractions (100 μl each) that passed through the column were combined and concentrated to the original 50 μl serum volumes by membrane filtration (Microcon YM-10, 10,000 MWCO, Millipore Corp., Bedford, Mass.). Based on an ELISA, more than 98-99% of the anti-fHbp antibodies were removed by the fHbp column.

Anti-fHbp antibody. ELISA was used to measure serum antibody titers to fHbp, which was performed as previously described (Welsch et al. J Immunol 2004; 172:5606-1). The solid-phase antigen consisted of rfHbp v.1 or v.2 proteins. The secondary antibody was a 1:2000 dilution of alkaline phosphatase-conjugated rabbit anti-mouse IgM+G+A (Zymed). The serum titer was defined as the dilution giving an OD₄₀₅ of 0.5 after 30-min incubation with substrate.

Complement-mediated bactericidal antibody activity. The bactericidal assay was performed as previously described (Moe G. R. et al, Infect Immun 2002; 70: 6021-31) using mid-log phase bacteria grown in Mueller Hinton broth supplemented with 0.25% glucose. The final reaction mixture contained different dilutions of test sera, and 20% (v/v) human complement. The buffer was Dulbecco's phosphate buffered saline (Sigma-Aldrich) containing 0.9 mM CaCl2×2H2O, 0.5 mM MgCl2×6H2O and 1% (w/v) BSA. The complement source was human serum from a healthy adult with no detectable intrinsic bactericidal activity (Granoff et al. J Immunol 1998; 160:5028-36; Welsch et al. 2003, supra). Serum bactericidal titers were defined as the serum dilution resulting in a 50% decrease in CFU per ml after 60 min. of incubation of bacteria in the reaction mixture, as compared with control CFU per ml at time 0. Typically, bacteria incubated with the negative control antibody and complement showed a 150 to 200% increase in CFU/mL during the 60 min. of incubation.

Binding of antibodies to the surface of live encapsulated N. meningitidis. The ability of anti-fHbp antibodies to bind to the surface of live N. meningitidis was determined by flow cytometric detection of indirect fluorescence assay, performed as described previously (Granoff et al. J Immunol 2001; 167:3487-3496). Positive controls included mouse monoclonal antibodies specific for the group C polysaccharide capsule (1076.1(Garcia-Ojeda et al. Infect Immun 2000; 68:239-46)), PorA P1.2 (Granoff et al. J Immunol 2001; 167:3487-3496), and fHbp variant 1 (JAR3)(Welsch et al. J Immunol 2004; 172:5606-15) and a 1:300 dilution of FITC conjugated Goat anti-mouse (Fab′)2 IgG (H+ L) (Jackson Immuno Research Laboratories, West Grove, Pa.).

Activation of human complement deposition on the surface of live encapsulated meningococci. Anti-fHbp antibody-dependent deposition of C3b or iC3b on the bacterial surface of live N. meningitidis bacteria was determined by flow cytometry, performed as previously described (Welsch et al. J Infect Dis 2003; 188:1730-40). Washed, log-phase bacteria were incubated in a reaction mixture containing 5% (v/v) human complement and appropriate serum dilutions in veranol buffer. Complement deposition was detected with FITC-conjugated sheep anti-human complement C3c (BioDesign Intl., Saco, Me.), which reacts with both C3b and iC3b. The complement source was the same human serum described above for the bactericidal assay.

### Example 1

### Development of Protective Neisseria Meningitidis Vaccine Having Reduced Endotoxicity

Vesicles vaccines prepared from mutant N. meningitidis strains engineered to over-express fHbp (OE fHbp), elicited broader protective serum antibody responses than certain control OMV or recombinant fHbp protein vaccines tested (Hou V. C. et al., J Infect Dis 2005; 192: 580-90 and Koeberling O. et al., Vaccine 2007; 25: 1912-20). The inventors sought to improve the toxicity profile of these “native” vesicle vaccines (i.e., vesicle vaccines that are not treated after production to reduce LPS, e.g., by detergent extraction) to provide for an improved safety profile for administration to humans.

Microvesicle (MV) vaccines were generated from mutant strains of N. meningitidis (H44/76 LpxL1 KO, referred to here as “LpxL1 KO” and NZ98/254 LpxL1KO). Both mutants contain structural changes in the lipopolysaccharide (LPS) molecule. The H44/76 mutant also had its endogenous fHbp gene inactivated and was engineered to over-express fHbp (gene from MC58) while endogenous expression of the fHbp gene was not interrupted in the NZ98/254 mutant, which was engineered to also express fHbp v.2 (gene from strain 8047).

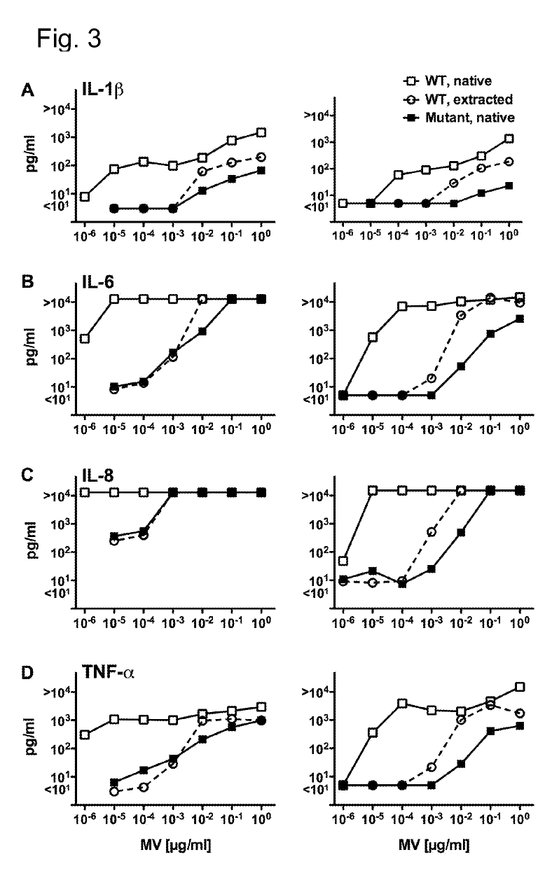

FIG. 1 summarizes the Meningococcal strains used in Examples 1-2. For these experiments, strain H44/76 and mutants derived from this strain were used to prepare the vesicle vaccines. This strain expresses a fHbp v.1 protein with an amino acid sequence identical to that of strain MC58 (Masignani V. et al., J Exp Med 2003; 197:789-99), which provided the gene to over-express fHbp v.1 (subvariant v.1.1). The other six strains expressed heterologous PorA proteins to that of the H44/76 vaccine strain and also expressed different subvariants of fHbp v.1. Microvesicle (MV) vaccines were generated from a mutant strain of N. meningitidis (H44/76 LpxL1 KO, referred to here as “LpxL1 KO”) that contains a structural change in the lipopolysaccharide (LPS) molecule. LpxL1 KO has the lpxL1 gene inactivated resulting in penta-instead of hexaacylated lipid A (FIG. 2). This mutant also had its endogenous fHbp gene inactivated and was engineered to express fHbp v.1 using the pFP12-fHbp shuttle vector (gene from MC58).

The LpxL1 KO was generated as described in materials and methods section. Insertional inactivation of the chromosomal lpxL1 gene was confirmed by PCR using LpxL1 specific primers and chromosomal DNA from the transformants as template. The sequences of the primers used, the sequence of the region deleted from LpxL1, and the upstream (5′) and downstream (3′) sequences flanking in the insertion in the LpxL1 gene are provided in the table below.

TABLE 1
Primer sequences:

Cloning of Lpxl1:
LpxL1_forHindIII:
cccaagcttgccgtctgaatcaatagtttcagacggc
(SEQ ID NO: 1)
LpxL1_revXbaI:
gctctagagccgtctgaacgtagtcagtaaaaatcggggc
(SEQ ID NO: 2)
Deletion of internal LPxL1 fragment:
LpxL1_del1:
aactgcagcggtgaagtgcggatacagg
(SEQ ID NO: 3)
LpxL1_del2:
acgcgtcgacaggatttcggacgcaacg
(SEQ ID NO: 4)
LpxL1 sequence deleted in plasmid pUClpxL1kan
Gttcgagatggcggtgtacgcgcttaatcaggatatcccgctgatcagta
tgtattcccatcaaaaaaacaagatattggAcgaacagattttgaaaggc
cgcaaccgctatcacaacgtcttccttatcgggcgcaccgaagggctgcg
cgccctcgtcaaacagttccgcaaaagcagcgcgccgtttctgtatctgc
ccgat
(SEQ ID NO: 5)
Upstream LpxL1 flanking region in plasmid
pUClpxL1kan
Tcaatagtttcagacggcatttgtattttgccgtctgaaaagaaaatgtg
tatcgagatgaaatttatattttttgtactgtatgtttTgcagtttctgc
cgtttgcgctgctgcacaagattgccgacctgacgggtttgcttgcctac
cttctggtcaaaccgcgccgcCgtatcggcgaaatcaatttggcaaaatg
tttttccgaatggagtgaggaaaagcgtaaaaccgtgttgaaacagcatt
tcaaAcacatggcgaaactgatgttggaatacggtttatattggtacgcg
cctgccggacgtttgaaatcgctggtgcgctaccgcaataagcattattt
ggacgacgcgctggcggcgggggaaaaagtcatcatcctgtatccgcact
tcaccgc
(SEQ ID NO: 6)
Downstream LpxL1 Flanking region in plasmid
pUClpxL1kan
Caggatttcggacgcaacgattcggtttttgtggattttttcggtattca
gacggcaacgattaccggattgagccgcattgccgcgCttgcaaatgcaa
aagtgatacccgccattcccgtccgcgaggcagacaatacggttacattg
catttctaccctgcttggaaatccTttccgggtgaagacgcgaaagccga
cgcgcagcgcatgaaccgttttatcgaagacagggtgcgcgaacatccgg
aacaatatttttggctgcacaagcgttttaaaacccgtccggaaggcagc
cccgatttttactgactacgt
(SEQ ID NO: 7)

The effect of the LpxL1 KO on LPS production was assessed by examining the LPS from microvesicles produced from wildtype H44/76 and from a LpxL1 KO strain overexpressing fHbp (LpxL1 OE fHbp) using silver-stained SDS PAGE. Samples were run at 20 mA constant for approximately 1.5 hours on a precast 4-20% Trix-glycine gradient gel (Invitrogen). The gel was agitated in 40% Ethanol, 5% acetic acid for 1 hour and treated for 5 min with 0.7% (w/v) periodic acid dissolved in 40% Ethanol, 5% acetic acid solution. After washing with H₂O (3×30 minutes) the gels were stained with 0.67% (w/v) silver nitrate in 0.019 M NaOH and 0.4% NH₄OH for 10 minutes and washed again with H₂O (3×10 min). The gels were developed in a solution containing 50 mg/l citric acid and 0.015% (v/v) formaldehyde. The reaction was stopped with 50% Methanol. As illustrated in FIG. 2, LPS from the LpxL1 OE fHbp strain migrated at a lower molecular weight, indicating that the LpxL1 knockout was effective in modifying LPS structure.

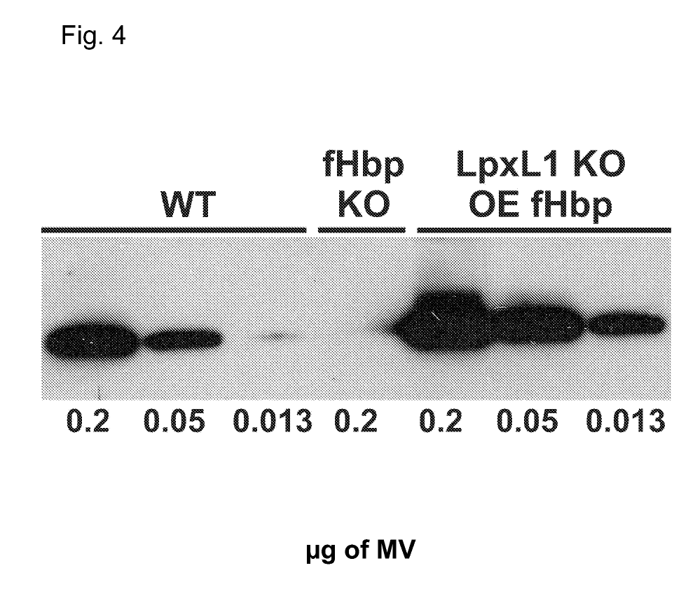

As a measure of toxicity, the ability of native MV vaccines (i.e., prepared without detergent extraction) prepared from the mutant and control wildtype (WT) parent strains to stimulate human peripheral blood mononuclear cells (PBMC) to produce 27 different cytokines were compared. Small doses of native MV from the wildtype strain elicited high levels of each of the cytokines while much higher doses of native MV prepared from the LpxL1 KO mutant with over-expressed fHbp, or the detergent-treated MV from the wildtype strain, were required for stimulation of cytokines. For example, with donor 2 (FIG. 3, Panel A, right), a dose of 4×10⁻⁶ μg/ml of native MV from the wildtype strain elicited the same concentration of TNF-α as did 3×10⁻² μg/ml of native mutant MV (ratio 1:7500), or 2.5×10⁻³ μg/ml of detergent-extracted wildtype MV (ratio 1:625). For each of the four cytokines shown in FIG. 3, the native MV from the mutant elicited lower or similar cytokine responses with both donor PBMCs as that of the detergent-extracted MV from the wildtype strain.

Of the 23 other cytokines measured, six (IL-1ra, G-CSF, IFN-γ, MCP-1, MIP-1α (and MIP-1β) were above background levels after incubation of PBMCs with OMV from the wildtype strain. For each of these cytokines the native OMV from the mutant had much lower stimulating activity than that of native OMV from the wildtype strain (>500- to 10.000-fold), and gave similar or lower stimulation as that of the detergent-extracted OMV from the wildtype strain (See Koeberling et al, J. Infect Dis. 2008). Collectively the data illustrate that a native MV vaccine would be suitable for administration to humans without the need for detergent treatments.

These data illustrate the development of a safe “native” MV and/or OMV vaccine formulation with over-expressed fHbp, which does not require treatment with detergents to decrease endotoxin activity while retaining immunogenicity. This vaccine approach could be combined with certain recombinant proteins to provide a universal vaccine for prevention of meningococcal disease.

### Example 2

### A MV Vaccine from a Mutant N. Meningitidis Strain with Over-Expressed fHbp and LpxL1 Inactivation Retains Immunogenicity

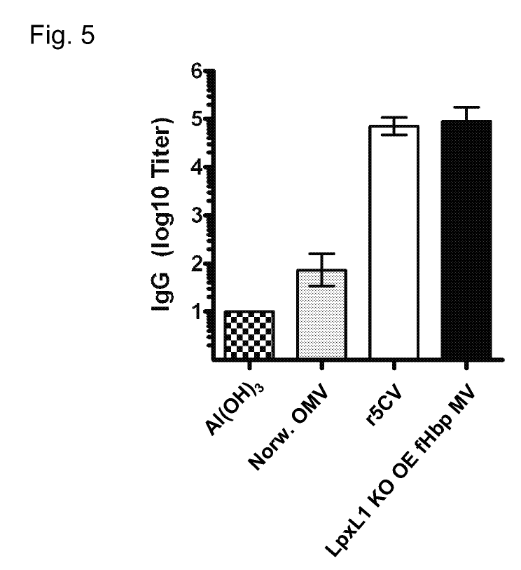

Relative amounts of fHbp in the different MV preparations were visualized by Western blot using a murine anti-fHbp mAb, JAR 3 (FIG. 4). In native MV prepared from the LpxL1 KO mutant with endogenous fHbp expression inactivated and fHbp v.1 expressed by the expression plasmid, 0.013 μg of protein contained slightly more fHbp than 0.05 μg of MV protein from the wildtype strain. Thus, the mutant MV contained approximately 4- to 5-fold more fHbp than the wildtype OMV.

The immunogenicity of native MV vaccines prepared from mutant strains of H44/76 with LpxL1 inactivated and fHbp over-expressed was investigated in an experiment involving more than 160 CD1 mice.

Groups of CD1 mice were immunized with 2 μg of a MV vaccine prepared from the LpxL1 KO with over-expressed fHbp. Control mice received adjuvant alone or 2 μg of the Norway detergent-treated OMV vaccine (Nokleby et al. Vaccine 2007; 25:3080-4; Bjune et al. NIPH Ann 1991; 14:125-30; discussion 130-2; Bjune et al. Lancet 1991; 338:1093-6; Holst et al. Vaccine 2003; 21:734-7) given with aluminum hydroxide, or 60 μg of a 5-component recombinant protein vaccine (r5CV) (Giuliani et al. Proc Natl Acad Sci USA 2006; 103:10834-9; Rappuoli, A universal vaccine for serogroup B Meningococcus. In: 15th International Pathogenic Neisseria Conference. Cairns, Australia, 2006) given with aluminium hydroxide. Groups of five negative control mice each received PBS or aluminium hydroxide, and groups of 10 to 15 mice each were assigned to different vaccine groups. Animals received three injections, each separated by three weeks, and were bleed 3 weeks after the last injections. Sera from groups of 5 animals each were pooled and assayed by ELISA for antibody titers against rfHbp v.1, and for bactericidal antibody using human complement. The results are reported as the geometric mean titers of three pools from each vaccine group (the only exceptions being the Norway OMV vaccine where there were two pools from 10 animals, and the negative control groups where one pool each was prepared from groups of five animals each receiving the different adjuvants).

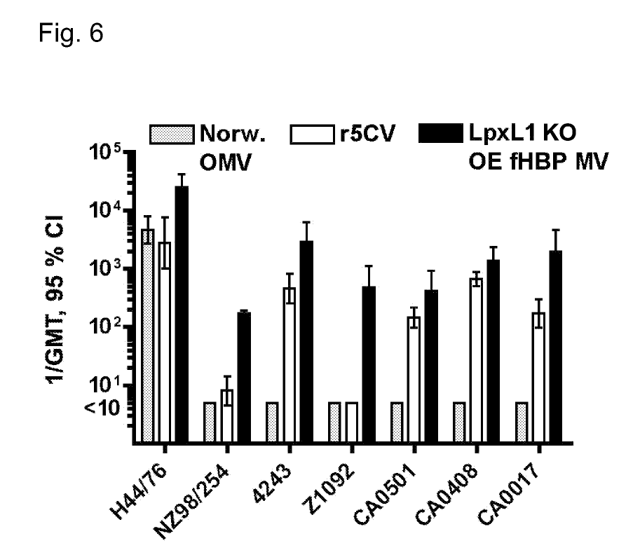

FIG. 5 summarizes the serum antibody responses to recombinant fHbp v.1. Mice immunized with the recombinant proteins had high serum antibody titers to fHbp. Mice immunized with the Norwegian OMV vaccine had low anti-fHbp titers (GMT of 1:80). In contrast, mice immunized with the native MV vaccine prepared from the LpxL1 KO mutant with over-expressed fHbp had high antibody responses to fHbp. The anti-fHbp GMT was 1:70,000, which was similar to that of the mice given the recombinant proteins (GMT of 1:100,000).

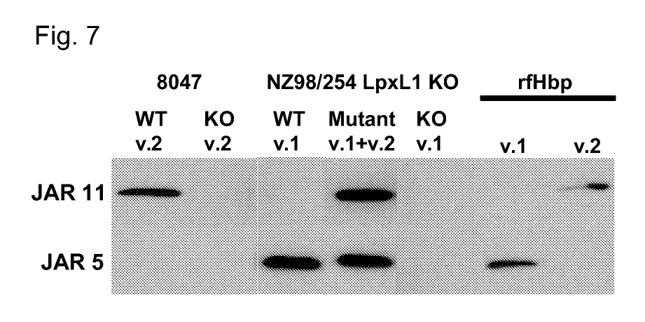

FIG. 6 summarizes the geometric means of the serum bactericidal titers as measured against seven test strains shown in FIG. 1. The titers of mice immunized with the aluminium hydroxide-absorbed MV vaccine prepared from the LpxL1 mutant with overexpressed fHbp (LpxL1 KO, OE fHbp) are compared to the respective titers of mice immunized with the 5C recombinant vaccine (r5CV) or the Norway detergent-extracted OMV vaccines, each administered with aluminium hydroxide. A table listing the characteristics of the test strains used as targets in the bactericidal assay is provided in FIG. 1.

The respective serum bactericidal titers of the different vaccine groups were similar against strain H44/76 (the parent strain from which the MV vaccines were prepared). However, against 6 strains tested with heterologous PorA to that of the strain used to prepare the vesicle vaccines, and expressing subvariants of v.1 fHbp, the responses of mice given the aluminium hydroxide-adsorbed MV vaccine with genetically detoxified endotoxin and over-expressed fHbp were higher than those of mice given the 5C or control Norway detergent-extracted OMV vaccines. The respective geometric mean titers against the six heterologous strains were 797 (mutant MV), 82 (5C) and <10 (Norway OMV).

### DISCUSSION

When measured against strain H44/76, the serum bactericidal responses of the mice given the native (H44/76 LpXL1 KO, OE fHbp) or detergent-treated vesicle vaccines (Norw OMV), or the recombinant 5C protein vaccine (r5CV), were high since this strain was used to prepare the vesicle vaccines, and also is known to be a high expresser of v.1 fHbp (data not shown). However, the results were quite different against six test strains with heterologous PorA to that of strain H44/76. These strains also express subvariants of variant 1 fHbp (FIG. 1). The sera from mice given the detergent-extracted OMV vaccine from Norway had no detectable bactericidal activity against any of the heterologous strains (titers <1:10) whereas the sera from mice immunized with the MV vaccine from the mutant with over-expressed fHbp had high titers against all six heterologous strains. The sera from the mice immunized with the recombinant 5C protein vaccine had high titers against some of the heterologous strains such as 4243 or CA0408 but had much lower titers (NZ98/254) or no detectable activity (Z1092) against other strains. For each of the six heterologous strains, the respective titers were higher in mice given the native MV vaccine from the mutant than the 5C recombinant protein or Norway OMV vaccines. The respective geometric mean titers were 1:787 (mutant MV), 1:82 (5C) and <1:10 (Norway OMV).

Immunogenicity. Both the recombinant 5C vaccine and the control Norway OMV vaccine have been tested in humans and have been shown to elicit serum bactericidal antibodies; the Norway OMV vaccine also been shown to confer protection against meningococcal disease during an epidemic in Norway. As compared with these two vaccines, this study shows significantly greater immunogenicity in mice of an investigational MV vaccine prepared from a LpxL1 KO mutant with over-expressed fHbp.

Safety. The cytokine stimulation data from human peripheral blood mononuclear exposed to a MV vaccine prepared from the LpxL1 mutant illustrates that overexpression of fHbp (fHbp) in combination with this mutant decreases LPS toxicity while retaining the ability to elicit protective antibodies against homologous and heterologous strains. Notably, the development of this native MV vaccine formulation does not require treatment with detergents to decrease endotoxin activity. Thus, vesicle vaccines prepared from fHbp-overexpressing, LpxL1 mutant strains without detergent extraction represents a good balance between decreasing lipid toxicity and preservation of immunogenicity.

A universal MenB vaccine. One attractive formulation for a universal meningococcal vaccine would be a combination of a native vesicle vaccine prepared from a LpxL1 mutant with over-expressed fHbp with the 5-component Norvartis recombinant protein vaccine with; or a combination of three recombinant proteins (fHbp v.1 or v.2; GNA2132 and NadA) “rC5”; see below).

A further attractive MV formulation would be a bivalent native MV vaccine from two different LpxL1 KO strains: NZ98/254 and H44/76, each engineered to over-express fHbp (subvariant v.1 from NZ98/254 and v.2 from 2996). MV vaccines from these mutants can be used together or combined with one or more recombinant proteins such as 287 or NadA. The mutant strains with over-expressed fHbp have been prepared and can be used to prepare native MV vaccines.

Still a further attractive MV formulation is a bivalent native MV vaccine from two mutant LpxL1 KO strains: NZ98/254 and H44/76, each engineered to over-express fHbp (subvariant v.1 from NZ98/254 and v.2 from 2996) (See below). MV vaccines from these mutants could be used together or combined with one or more recombinant proteins such as 287 or NadA. The mutant strains with over-expressed fHbp have been prepared and could be used to prepare native MV vaccines for this study. In addition, a bivalent native OMV vaccine composed of two mutant LpxL1 KO strains (e.g., NZ98/254 and H44/76) each engineered to over-express one or more fHbps also provides an attractive vaccine. OMV vaccines from these mutants can optionally be combined with one or more recombinant proteins, which may be either expressed in one or both of the strains or provided in isolated form.

### Example 3

### Broad Immunity Elicited by Native MV Vaccine from Mutant N. Meningitidis Strains

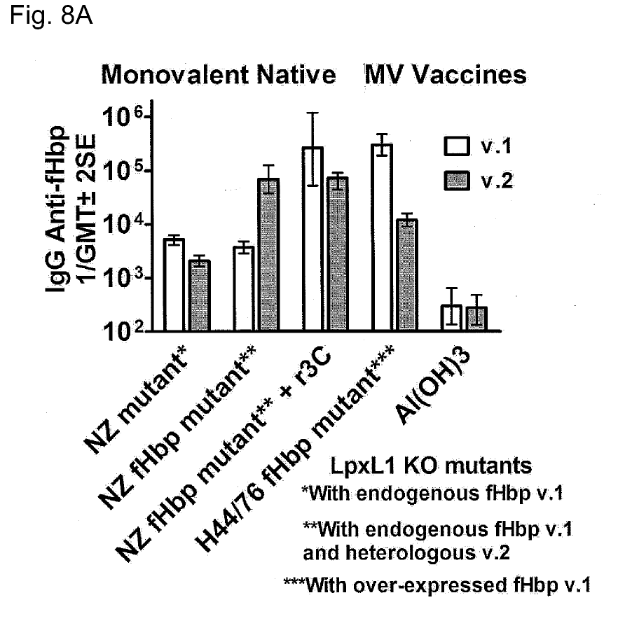

The LpxL1 knockout (KO) mutant of strain NZ98/254 was engineered to express heterologous fHbp v.2 (gene from strain 8047) using plasmid pComP1523. The fHbp gene integrated into a non-coding region of the N. meningitidis chromosome between nmb1428 and nmb1429 under control of the strong promoter from gene nmb1523. Since the endogenous fHbp gene from NZ98/254 (subvariant of fHbp v.1) was not inactivated, the NZ98/254 mutant co-expressed endogenous fHbp v.1 and heterologous v.2 (FIG. 7, MV from “mutant v.1+v.2”, Western blot).

As described previously in Examples 1 and 2 for the vaccine from the H44/76 LpxL1 KO, incubation of the MV vaccines from the LpxL1 KO mutants of NZ98/254 with human PBMCs elicited similar concentrations of proinflammatory cytokines as those elicited by a detergent-treated MV vaccine prepared from the wildtype strain of NZ98/254 (data not shown). Since similar cytokine-release data are being used to replace the rabbit pyrogenicity test to predict human responses to pyrogens (Nakagawa Y. et al., Clin Diagn Lab Immunol 2002; 9:588-97), these data indicate that native OMV vaccine from the NZ98/254 LpxL1 KO mutant will be well-tolerated in humans.

The immunogenicity of native MV vaccine prepared from mutant group B NZ98/254 strain with LpxL1 inactivated and expressing endogenous fHbp v.1 and heterologous fHbp v.2 was investigated in mice. The MV vaccine was given alone, or as a bivalent MV vaccine with a second LpxL1 KO strain made from the strain H44/76 with endogenous fHbp inactivated and further engineered to overexpress fHbp v.1 (H44/76 LpxL1 KO, OE fHbp v.1). MV from these strains that were not subjected to detergent treatment are referred to as “native” MV for the purposes of these examples. A vaccine with MV from both of these strains is referred to in these examples as a “bivalent” vaccine. A third combination formulation (r3C) consisted of the MV from the NZ98/254 mutant combined with three recombinant proteins, fHbp v.1 (gene from strain MC58), GNA2132 (gene from strain NZ98/254) and NadA (gene from strain 2996) was tested.

TABLE 2
Summary of strains with expressed heterologous fHbp

| Vaccine | | PorA VR | WT³ | WT fHbp | Expressed fHbp | |
|---|---|---|---|---|---|---|
| Strain | ST Complex¹ | Type² | fHbp | KO | (Plasmid) | LPS |
| RM1090 | Not Done | 5-1,2 | v.2 sv⁴ | Yes | v.1 (pFP12-fHbp) | WT |
| H44/76 | 32 | 1.7,16 | v.1 | Yes | v.1 (pFP12-fHbp) | WT |
| H44/76^{†} | 32 | 1.7,16 | v.1 | Yes | v.1 (pFP12-fHbp) | LpxL1 KO |
| NZ98/254 | 41/44 | 1.7-2,4 | v.1 sv | Yes | v.1 sv (pComP1523) | WT |
| NZ98/254^{†} | 41/44 | 1.7-2,4 | v.1 sv | No | v.2 sv (pComP1523) | LpxL1 KO |
| NZ98/254 | 41/44 | 1.7-2,4 | v.1 sv | Yes | v.2 sv (pComP1523) | LpxL1 KO |

¹Sequence type complex.
²PorA variable region sequence type.
³WT, wildtype
⁴Sv., sub-variant of fHbp.
^{†}strains used to prepare native MV vaccines described herein

Table 2 shows the different N. meningitidis group B strains from different genetic lineages used to prepare mutants with increased expression of fHbp. One approach was first to inactivate the endogenous fHbp gene, and then transform the KO with an expression vector, such as pFP12-fHbp, pComP1523 (Table 2) or pComPind (not shown), to generate second-generation mutants with over-expressed fHbp v.1. The vector, pFP12fHbp, was not integrated into the chromosome and regulated expression of the fHbp gene with its own promoter.

A second approach is illustrated by a mutant of strain NZ98/254 in which fHbp v.2 was expressed by integrating pComP1523 in a non-coding region of the N. meningitidis chromosome without inactivation of the endogenous subvariant (sv) 1 fHbp gene, which permitted co-expression of endogenous fHbp v.1 and heterologous fHpb v.1 (FIG. 7).

When transformation was done with each of the different plasmids using a naturally high fHbp-expressing strain, H44/76, approximately 4- to 5-fold increased expression of fHbp was achieved as compared with that of the respective wildtype (WT) strain. When transformations were done in naturally low expressing strains, even higher relative increases in fHbp expression was observed. In all of the mutants tested to date, fHbp was present on the surface as measured by anti-fHbp mAb binding by flow cytometry with log phase, live encapsulated bacteria (Hou V. C., et al., 2005, supra; Koeberling O., 2007, supra). The increases in the relative expression of fHbp resulted in MV vaccines that elicited much higher anti-fHbp bactericidal antibody responses than elicited by control MV vaccines prepared from the respective wildtype strains or recombinant fHbp vaccines (Hou V. C., et al., 2005, supra; Koeberling O., 2007, supra). Based on SDS-PAGE, expression of the major MV proteins, including PorA, PorB, and Rmp, was similar in the respective parent and mutant MV vaccines.

Native MV vaccines from the LpxL1 KO mutants were prepared by isolation of membrane blebs released by bacteria grown in broth culture using procedures previously described for preparation of MV vaccines from other strains (Moe G. R. et al, Infect Immun 2002; 70: 6021-31). The immunogenicity of native MV vaccines was investigated in mice. A monovalent MV vaccine (prepared from strain NZ98/254 LpxL1 KO, endogenous fHbp v.1, heterologous fHbp v.2) given alone (dose of 2.5 μg of protein), or given in combination with a native MV vaccine prepared from the LpxL1KO mutant of H44/76 with over-expressed fHbp v.1 (H44/76 LpxL1KO, OE fHbp v.1) (Koeberling O. et al., J Infect Dis 2008; 198:262-70), or given in combination with three recombinant proteins fHbp v.1 (gene from strain MC58), GNA2132 (gene from strain NZ98/254) and NadA (gene from strain 2996) was tested. The combination MV vaccine is referred to as “bivalent MV vaccine”, 2.5 μg of each MV, or total dose of 5 μg). The combination vaccine of monovalent MV vaccine (prepared from strain NZ98/254 LpxL1 KO, endogenous fHbp v.1, heterologous fHbp v.2) and recombinant 3C vaccine is called monovalent MV vaccine+r3C (2 dose of 5 μg of the MV and dose of 20 μg of each recombinant protein, or a total dose 60 μg of recombinant proteins). Mice (10 to 15 per group) were given three injections of vaccine, IP, each separated by 3 weeks. All vaccine antigens were adsorbed with aluminum hydroxide as an adjuvant (total dose per injection, 600 μg). Blood was obtained 3 weeks after dose 3. Serum pools were prepared from blood of 4 to 5 mice (2 to 3 pools per vaccine groups) and were assayed for IgG antibody responses to recombinant fHbp v.1 or v.2 by ELISA, performed as previously described. Bactericidal activity was measured in the serum pools with human complement, and was performed as previously described. For the test strains for the bactericidal assay, a panel of N. meningitidis strains was used (Table 3). The strains in this panel express PorA homologous or heterologous to those of the two vaccine strains used to prepare the MV vaccines, and express fHbp in the variant 1, 2, or 3 groups).

TABLE 3
Neisseria meningitidis strains (Europe and U.S) tested for SBA

| | | fHbp | | |
|---|---|---|---|---|
| | Capsular | Variant | PorA | Sequence |
| Strain | Group | Group | VR Sequence Type | Type |
| Vaccine WT | | | | |
| strains | | | | |
| H44/76^{†} | B | 1 | 1.7, 16 | 32 |
| NZ98/254^{†} | B | 1 | 1.7-2, 4 | 42 |
| Heterologous | | | | |
| strains (PorA) | | | | |
| Z1092* | A | 1 | 1.5-2, 10 | 11 |
| 4243* | C | 1 | 1.5, 2 | 11 |
| M2197* | C | 1 | 1.7, 1 | 11 |
| LNP 17592* | W-135 | 1 | 1.5, 2 | 11 |
| GB200* | B | 1 | 1.22, 9 | 275 |
| 8047** | B | 2 | 1.5-1, 2-2 | 8 |
| GB013* | B | 2 | 1.22, 9 | 275 |
| MD1248* | B | 2 | 1.22, 26 | 44 |
| 2996** | B | 2 | 1.19, 15 | 8 |
| M1239* | B | 3 | 1.23, 14 | 41/44 |
| 03S-0451* | B | 3 | 1.22-1, 14 | 32 |

^{†}Vaccine strains from which mutants were made for MV vaccines.
*Strains with heterologous PorA to those of vaccine strains H44/76 and NZ98/254 and expressing subvariants of fHbp v.1 or v.2, or fHbp v.3
**Strains expressing fHbp v.2 >99.5% identical to that expressed by mutant of NZ98/254 with heterologous v.2. Note strain 2996 is low expresser of fHbp

Results

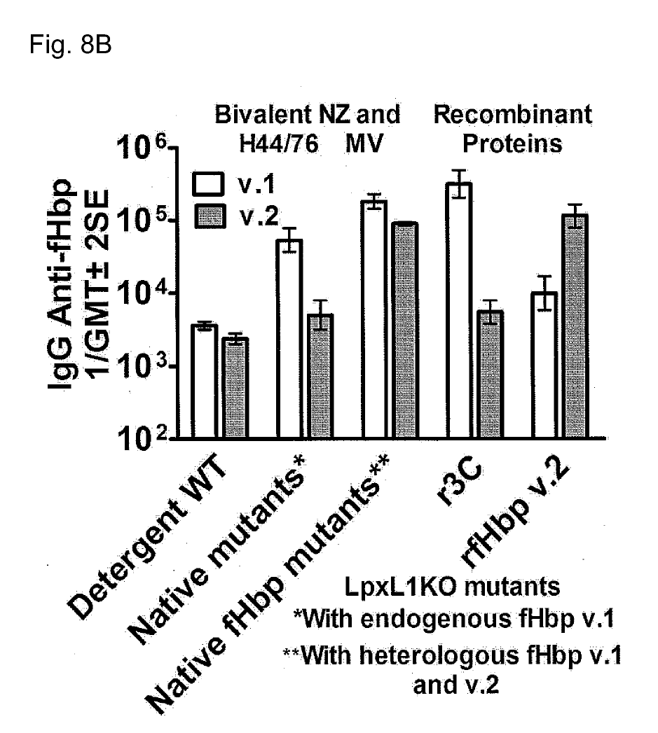

IgG anti-fHbp antibody responses. Control mice immunized with a monovalent detergent-treated MV vaccine from the wildtype strain of NZ98/254, or a native MV vaccine prepared from LpxL1 KO mutant of strain NZ98/254 that expressed only endogenous fHbp v.1, showed low IgG anti-fHbp antibody responses to both the variant 1 or 2 proteins (data not shown and FIG. 8A). In contrast, mice immunized with the corresponding native MV vaccine prepared from the LpxL1KO mutant of NZ98/254 that expressed both endogenous fHbp v.1 and heterologous fHbp v.2 showed nearly 100-fold higher antibody responses to fHbp v.2 than the control mice. However, unless the native NZ98/254 MV with endogenous fHbp v1. and heterologous fHbp v.2 was given in combination with the recombinant protein vaccine (3C), it did not elicit high responses to fHbp v.1. In contrast, mice immunized with a native MV vaccine prepared from the LpxL1 KO mutant of strain H44/76 with over-expressed fHbp v.1 had high IgG antibody responses to fHbp v.1, but not to v.2 (FIG. 8A).

Mice immunized with the native MV vaccine from the NZ98/254 fHbp mutant given in combination with the recombinant 3C vaccine (FIG. 8A), or with a bivalent native MV vaccine prepared from NZ98/254 LpxL1 KO, endogenous fHbp v.1 and heterologous fHbp v.2 and H44/76 LpxL1 KO, fHbp KO, expressing fHbp v.1 from heterologous promoter (FIG. 8B) showed high IgG antibody responses to both fHbp v1. and v.2. Interestingly, control mice immunized with a bivalent OMV vaccine prepared from the LpxL1 KO mutants of strains NZ98/254 and H44/76 that only expressed the respective endogenous fHbps in the v.1 group showed relatively high anti-fHbp antibody responses to the v.1 protein (FIG. 8B). This result likely reflected the combined immunogenicity of naturally high levels of endogenous fHbp v.1 in the MV from the H44/76 strain, together with low expression of endogenous subvariant of fHbp v.1 by the NZ98/254 strain.

Serum bactericidal antibody responses. Serum bactericidal antibody responses were measured against a panel of genetically diverse N. meningitidis strains (shown in Tables 3 and 4).

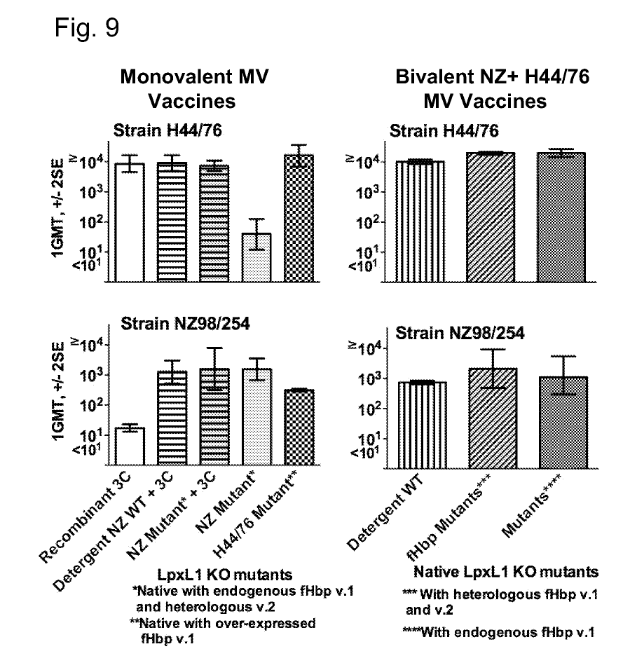

Bactericidal activity against the two wildtype strains used to prepare mutants for MV vaccines. Mice immunized with the monovalent MV vaccines developed high serum bactericidal antibody responses against the respective homologous wildtype test strains from which the respective mutants were derived to prepare the vaccines (FIG. 9). Mice immunized with native MV vaccine from the H44/76 LpxL1 KO, fHbp KO, expressing fHbp v.1 from heterologous promoter also had high bactericidal titers against the heterologous NZ98/254 strain (FIG. 9, lower left panel). Mice immunized with the bivalent native MV from the mutants, or detergent-treated MV from the corresponding wildtype strains had high bactericidal titers against both of the test strains with homologous PorA.

Note that detergent-treated bivalent meningococcal MV vaccines, including a vaccine prepared from strains NZ98/254 and H44/76, have been investigated in humans and also elicited serum bactericidal antibodies against the homologous strains used to prepare the vaccines (Sandbu S. et al., CVI 2007, 14 (9): 1062-1069).

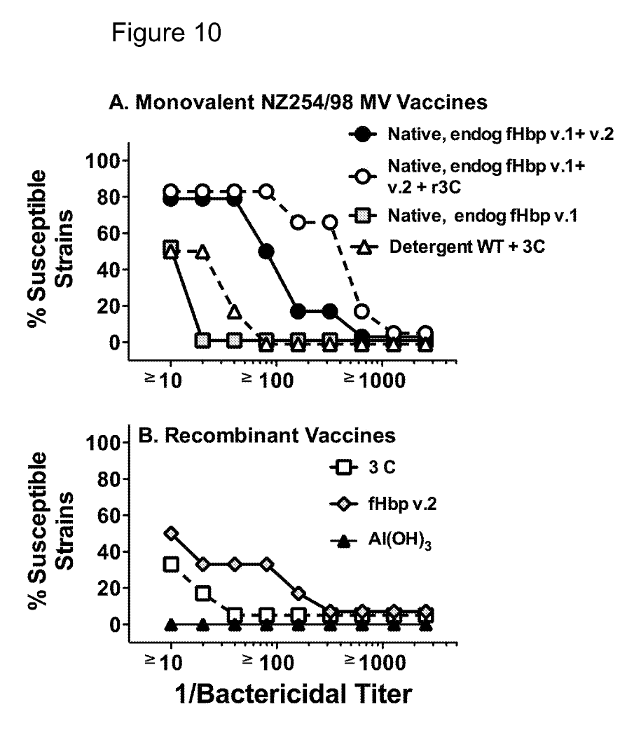

Bactericidal responses of mice immunized with the monovalent native MV from NZ98/254 LpxL1 KO, endogenous fHbp v.1 and heterologous fHbp v.2 strain. The serum bactericidal responses of mice immunized with the MV vaccine from the mutant of NZ98/254 measured against six group B strains with heterologous PorA to those of the vaccine strains and expressing fHbp in the v.2 or v.3 groups are shown in FIG. 10. A strain was considered susceptible to bactericidal activity if the CFU/ml of bacteria decreased by 50 percent after incubation for one hr at 37 degrees C. with 20 percent human complement and the dilution of the test sera (as compared to CFU/ml present at time 0 in negative control sera). Over 80 percent of the strains tested (5/6 strains, Panel A) were susceptible to bactericidal activity of sera from mice immunized with the MV prepared from the native MV from NZ98/254 LpxL1 KO, endogenous fHbp v.1 and heterologous fHbp v.2 strain when the sera diluted up to 1:30, and half of the strains were susceptible at dilutions up to 1:90. Much lower proportions of strains were susceptible to bactericidal activity of these dilutions of sera from control mice immunized with detergent-treated MV vaccine from the wildtype NZ98/254 strain, or a native MV from the LpxL1 KO mutant of NZ98/254 that only expressed had endogenous fHbp v.1.

Only a small proportion of the strains were susceptible to bactericidal activity of sera from mice immunized with the recombinant 3C vaccine (Panel B). However, mice immunized with the r3C vaccine combined with the MV vaccine from the NZ98/254 LpxL1 KO with endogenous fHbp v.1 and heterologous fHbp v.2, had highest titers against this panel of heterologous group B strains that expressed fHbp in the v.2 or v.3 antigenic groups.

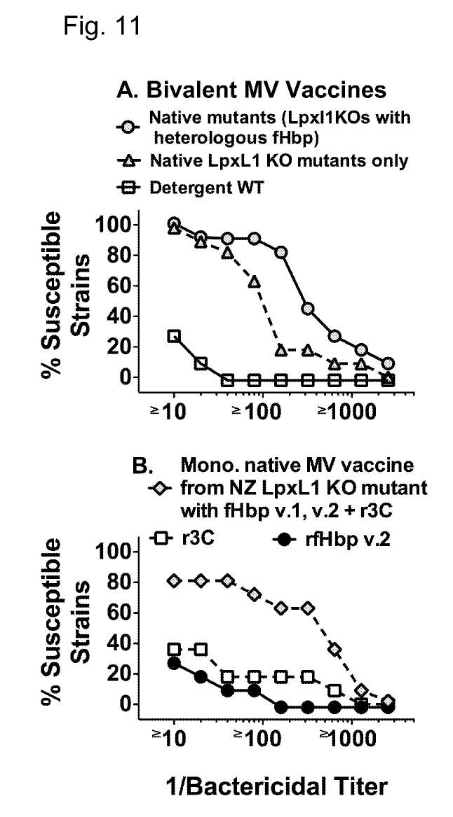

Bactericidal responses of mice immunized with a bivalent native MV vaccine. The serum bactericidal responses of mice immunized with bivalent MV vaccines were measured against 11 test strains from Europe or the United States that had heterologous PorA to those to the two vaccine strains and fHbps in the v.1, 2 or 3 groups (FIG. 11, Panel A). The test strains in this analysis included all the heterologous strains shown in Table 3 (i.e., the susceptible homologous H44/76 and NZ98/254 test strains which were used to prepare the mutants for the MV vaccines, were not excluded). The bivalent native MV vaccine prepared from the LpxL1 KO mutants with heterologous fHbp (i.e., NZ98/254 LpxL1 KO with both endogenous fHbp v.1 and heterologous v.2, and H44/76 LpxL1 KO with endogenous fHbp inactivated and expressing fHbp v.1 from a heterologous promoter) elicited bactericidal activity against all 11 strains with titers >1:200 against >80 percent of the strains. The control detergent-treated MV vaccine from the two corresponding wildtype strains elicited bactericidal activity against less than 30 percent of the strains and with much lower titers (Panel A). The control recombinant protein fHbp v.2 vaccine and the recombinant 3C vaccine also elicited low titers against less than half of the strain (Panel B). However, the combination vaccine consisting of the native monovalent MV from the NZ254/98 LpxL1 KO with endogenous fHbp v.1 and heterologous fHbp v.2 strain plus r3C elicited high titers against 80 percent of the strains.

The bivalent native MV vaccine prepared from mutants of strain NZ98/254 with LpxL1 inactivated and only endogenous fHbp v.1 and strain H44/76 with LpxL1 inactivated and only endogenous fHbp v.1) elicited relatively high titers against each of the strains (FIG. 11, Panel A). Thus, bivalent native MV vaccine prepared from the LpxL1 KO mutants that only expressed endogenous fHbp in the variant 1 group was remarkably better than bivalent detergent treated MV vaccine prepared from the wildtype strains that only expressed endogenous fHbp in the variant 1 group (FIG. 11, Panel A). This high and broad bactericidal activity against heterologous test strains is opposite from that observed in a previous study of mice and guinea pigs immunized with a native trivalent OMV vaccine prepared from three other N. meningitidis wildtype strains (BZ198, RM1090, Z1092) (Moe G. R. et al, Infect Immun 2002; 70: 6021-31). The breadth of the protective antibody responses of mice observed in the present study immunized with the bivalent native MV vaccine from the H44/76 LpxL1 KO and NZ98/254 LpxL1 KO strains also would not be predicted based on the responses to each of the respective monovalent MV vaccines, which were low to most heterologous strains that expressed fHbp v.2 or v.3 (FIG. 10, Panel A for results of mice immunized with the monovalent native MV from the NZ98/254 expressing only endogenous fHbp v.1). Without being held to theory, the bivalent native MV vaccine prepared from these strains appears to contain unique combinations of antigens other than fHbp, which together are capable of eliciting broadly protective serum antibody responses. Albeit not as broad or high as the bivalent native MV vaccine prepared from the corresponding LpxL1 KO mutants with heterologous fHbp v.1 and v.2 expression (FIG. 11, Panel A), or a combination vaccine of the monovalent native MV from the LpxL1 KO mutant of NZ98/254 expressing endogenous fHbp v.1 and heterologous fHbp v.2 mixed with three recombinant protein antigens (r3C) (FIG. 10, Panel B). All three of these native MV vaccines, or combinations, elicited significantly higher and broader serum bactericidal antibody responses against genetically diverse N. meningitidis strains expressing fHbp v.1, 2 or 3 than vaccines consisting of a bivalent detergent-treated MV vaccine from the corresponding wildtype strains (FIG. 10, Panel A), or a recombinant 3C vaccine, or a recombinant fHbp v.2 vaccine (FIG. 10, Panel B).

### Example 4

### Immunity Against Epidemic Strains from Africa with Capsular Groups A, W-1350or X Elicited by a Native MV Vaccine from Mutant N. Meningitidis Strains

The bivalent MV vaccines described in Example 3 were also tested against epidemic strains from Africa with capsular groups A, W-135 and X (Strains listed in Table 4).

TABLE 4
N. meningitidis strains from Africa

| | | Capsular | | fHbp | |
|---|---|---|---|---|---|
| Strain | Country | Group | ST | Variant | PorA |
| LNP20290 | Burkina Faso | A | 2859 | 1 | P1.9 |
| E23/03 | Ethiopia | A | 7 | 1 | P1.20,9 |
| F6142 | Chad | A | 5 | 1 | P1.20,9 |
| M9262 | Burkina Faso | W-135 | 11 | 2 | P1.5,2 |
| Mali 29/07 | Mali | W-135 | 11 | 1 | P1.5,2 |
| Su 1/06 | Sudan | W-135 | 11 | 1 | P1.5,2 |
| Ug 13/07 | Uganda | X | 5403 | 1 | P1.19,26 |
| BuFa 7/07 | Burkina Faso | X | 181 | 1 | P1.5-1,10-1 |

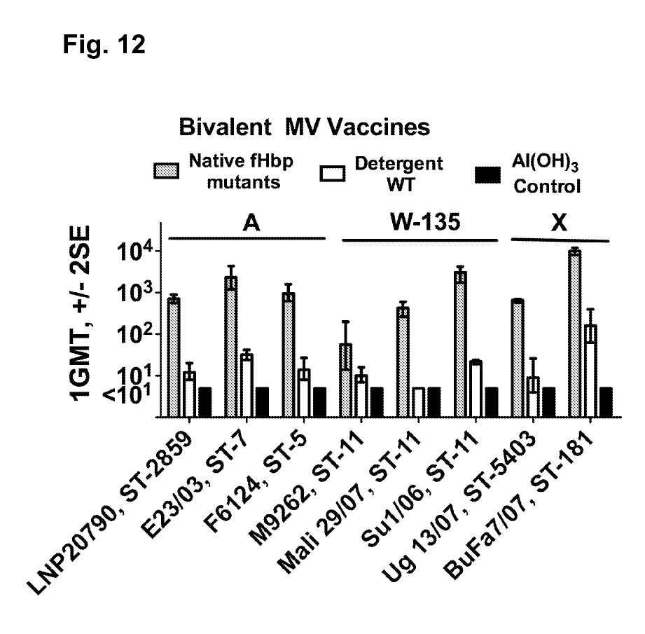

These strains are responsible for tens of thousands of cases in Sub-Saharan African but rarely cause invasive meningococcal disease in the U.S. or Europe. FIG. 12 shows serum bactericidal antibody responses against strains with capsular groups A, W-135 or X. All of the strains have heterologous PorA to those of the strains used to prepare the MV vaccine. With one exception the strains from Africa express subvariants of fHbp in the v.1 group. The exception was the W-135 strain from Burkina Faso that expressed a subvariant of fHbp in the v.2 group. The bivalent vesicle vaccine (from strain H44/76 LpxL1 KO, OE fHbp v.1 and NZ98/254 LpxL1 KO, endogenous fHbp v.1 and heterologous fHbp v.2) elicited high serum bactericidal antibody responses against all of the strains tested, whereas the control detergent-treated bivalent MV vaccine from the two wildtype strains of H44/76 and NZ98/254 elicited relatively low bactericidal responses except to one of the group X strains. Thus, a bivalent MV vaccine from LpxL1KO mutant strains engineered to express heterologous fHbps could serve as a universal meningococcal vaccine to control epidemic disease in Africa caused by strains from each of the capsular groups responsible for epidemic disease in that region.

### Example 5

### Preparation of Vaccines from Mutant Strains Belonging to Capsular Groups Other than Group B

Mutants are prepared from selected wildtype group A, W-135, and/or X strains. Certain strains derived from Africa are of particular interest. These mutants are engineered to over-express heterologous or homologous fHbps using methods described previously. At least one of the strains is selected for naturally high expression of NadA. The LpxL1 genes of the strains are also inactivated to attenuate endotoxin activity using methods described in Example 1. The selected strains may express one or more variants or subvariants of fHbps in the same time.

Vesicles can be purified from these strains without the use of detergents to maximize retention of desirable antigens. These “native” vesicles are administered to human peripheral blood mononuclear cells to assess toxicity by measuring in vitro concentration-dependent cytokine responses. Immunogenicity can be confirmed in mice and serum bactericidal antibody responses measured against a panel of strains, including strains representatives of each of the different capsular groups.

What is claimed is:

1. A composition comprising:
isolated antigenic vesicles prepared from a first Neisseria bacterium, wherein the Neisseria bacterium is genetically modified to
a) provide for decreased or no activity of a gene product of the lpxL1 gene; and
b) express a heterologous fHbp polypeptide;
an isolated Neisserial antigen; and
a pharmaceutically acceptable carrier.

2. The composition of claim 1, wherein the isolated Neisserial antigen comprises fHbp, GNA2132 and Nad A polypeptides.

3. The composition of claim 2, wherein the isolated Neisserial antigen further comprises GNA2091 and GNA1030 polypeptides.

4. The composition of claim 2, wherein the heterologous fHbp is fHbp v.2.

5. The composition of claim 3, wherein the heterologous fHbp is fHbp v.2.

6. The composition of claim 2, wherein the first Neisseria bacterium is NZ98/254.

7. The composition of claim 3, wherein the first Neisseria bacterium is NZ98/254.

8. The composition of claim 1 further comprising:
isolated antigenic vesicles prepared from a second Neisseria bacterium genetically diverse to the first Neisseria bacterium, wherein the second Neisseria bacterium is genetically modified to
a) provide for decreased or no activity of a polypeptide product of the lpxL1 gene; and
b) provide for decreased or no production of an endogenous fHbp polypeptide; and
c) express a recombinant fHbp polypeptide.

9. The composition of claim 8, wherein the isolated Neisserial antigen comprises fHbp, GNA2132 and Nad A polypeptides.

10. The composition of claim 9 wherein the isolated Neisserial antigen further comprises GNA2091 and GNA1030 polypeptides.

11. The composition of claim 8, wherein the recombinant fHbp polypeptide is expressed from a construct comprising a nucleic acid encoding a fHbp polypeptide operably linked to a heterologous promoter.

12. The composition of claim 8, wherein the recombinant fHbp polypeptide is of the same variant group as fHbp polypeptide endogenous to the second Neisseria bacterium.

13. The composition of claim 9, wherein the first Neisseria bacterium is NZ98/254 and the second Neisseria bacterium is H44/76.

14. The composition of claim 10, wherein the first Neisseria bacterium is NZ98/254 and the second Neisseria bacterium is H44/76.

15. A composition comprising:
isolated antigenic vesicles prepared from a first Neisseria bacterium that is genetically modified to provide for decreased or no activity of a polypeptide product of the lpxL1 gene;
isolated antigenic vesicles prepared from a second Neisseria bacterium that is genetically modified to provide for decreased or no activity of a polypeptide product of the lpxL1 gene and is genetically diverse to the first Neisseria bacterium; and
a pharmaceutically acceptable carrier.

16. The composition of claim 15 further comprising Nesisserial antigens comprising fHbp, GNA2132 and Nad A polypeptides.

17. The composition of claim 16 further comprising Nesisserial antigens comprising GNA2091 and GNA1030 polypeptides.

18. The composition of claim 16, wherein the first Neisseria bacterium is NZ98/254 and the second Neisseria bacterium is H44/76.

19. The composition of claim 17, wherein the first Neisseria bacterium is NZ98/254 and the second Neisseria bacterium is H44/76.

20. The composition of claim 15, wherein
the first Neisseria bacterium is genetically modified to express a heterologous fHbp polypeptide; and
the second Neisseria bacterium is genetically modified to provide for decreased production of endogenous fHbp polypeptide and to provide for expression of a recombinant fHbp polypeptide.

21. The composition of claim 20, wherein the recombinant fHbp polypeptide is expressed from a construct comprising a nucleic acid encoding a fHbp polypeptide operably linked to a heterologous promoter.

22. The composition of claim 20, wherein the recombinant fHbp polypeptide is of the same variant group as fHbp polypeptide endogenous to the second Neisseria bacterium.

23. A method of eliciting an immune response against Neisseria, said method comprising the steps of:
administering to a mammal an immunologically effective amount of the composition of claim 1;
wherein said administering is sufficient to elicit an immune response to a fHbp polypeptide present in the composition.

24. A method of eliciting an immune response against Neisseria, said method comprising the steps of:
administering to a mammal an immunologically effective amount of the composition of claim 15;
wherein said administering is sufficient to elicit an immune response to a fHbp polypeptide present in the composition.

25. A method of producing the antigenic composition of claim 1, the method comprising:
culturing the first Neisseria bacterium;
preparing isolated vesicles from the cultured bacterium; and
combining the isolated vesicles with the isolated Neisserial antigen and a pharmaceutically acceptable carrier;
wherein an antigenic composition is produced.

26. A method of producing the antigenic composition claim 15, the method comprising:
culturing the first Neisseria bacterium and the second Neisseria bacterium; and
preparing vesicles from the cultured first Neisseria bacterium and second Neisseria bacterium;
combining the vesicles with a pharmaceutically acceptable carrier;
wherein an antigenic composition is produced.

27. The method of claim 25, wherein the isolated Nesisserial antigen comprises fHbp, GNA2132 and Nad A.

28. The method of claim 27, wherein the isolated Nesisserial antigen further comprises GNA2091 and GNA1030 polypeptides.

29. The method of claim 26, wherein the first Neisseria bacterium is NZ98/254 and the second Neisseria bacterium is H44/76.

30. The method of claim 26, wherein
the first Neisseria bacterium is genetically modified to express a heterologous fHbp polypeptide; and
the second Neisseria bacterium is genetically modified to provide for decreased production of endogenous fHbp polypeptide and to provide for expression of a recombinant fHbp polypeptide.

31. The method of claim 30, wherein the recombinant fHbp polypeptide is expressed from a construct comprising a nucleic acid encoding a fHbp polypeptide operably linked to a heterologous promoter.

32. The method of claim 30, wherein the recombinant fHbp polypeptide is of the same variant group as fHbp polypeptide endogenous to the second strain.


SEQUENCE LISTING (9 sequences)

SEQ ID NO: 1 - DNA, 29 bp, Artificial Sequence
  oligonucleotide
cccaagcttatccttcggggatgcaggtc

SEQ ID NO: 2 - DNA, 40 bp, Artificial Sequence
  oligonucleotide
gctctagagccgtctgaacgtagtcagtaaaaatcggggc

SEQ ID NO: 3 - DNA, 28 bp, Artificial Sequence
  oligonucleotide
aactgcagcggtgaagtgcggatacagg

SEQ ID NO: 4 - DNA, 28 bp, Artificial Sequence
  oligonucleotide
acgcgtcgacaggatttcggacgcaacg

SEQ ID NO: 5 - DNA, 205 bp, Neisseria meningitidis
gttcgagatggcggtgtacgcgcttaatcaggatatcccgctgatcagtatgtattcccatcaaaaaaacaagatattggacgaacagattttgaaaggccgcaaccgctatcacaacgtcttccttatcgggcgcaccgaagggctgcgcgccctcgtcaaacagttccgcaaaagcagcgcgccgtttctgtatctgcccgat

SEQ ID NO: 6 - DNA, 407 bp, Neisseria meningitidis
tcaatagtttcagacggcatttgtattttgccgtctgaaaagaaaatgtgtatcgagatgaaatttatattttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgc

SEQ ID NO: 7 - DNA, 321 bp, Neisseria meningitidis
caggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgt

SEQ ID NO: 8 - DNA, 29 bp, Neisseria meningitidis
atcggcatgcgccgttcggacgacatttg

SEQ ID NO: 9 - DNA, 30 bp, Neisseria meningitidis
aagaaggcctttattgcttggcggcaaggc